(19) United States
(12) Patent Application Publication
(10) Pub. No.: US 2010/0112100 A1
(43) Pub. Date: May 6, 2010

(54) COMPOSITIONS COMPRISING A C-GLYCOSIDE COMPOUND

(75) Inventors: Claudie WILLEMIN, Paris (FR); Laure Bissey, Levallois-Perret (FR); Jerome Senee, Lardy (FR); Delphine Compain, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/685,285

(22) Filed: Jan. 11, 2010

Related U.S. Application Data
(63) Continuation of application No. 12/045,767, filed on Mar. 11, 2008, now abandoned.
(60) Provisional application No. 60/894,254, filed on Mar. 12, 2007.

(51) Int. Cl. A61K 8/49; A61K 8/97; A61K 8/99

(52) U.S. Cl. 424/732; 514/23; 424/725; 424/756; 514/12; 514/54; 424/780

(57) ABSTRACT

The present invention relates, in particular, to:
novel uses of a C-glycoside compound;
the use, in a cosmetic composition in particular, of a C-glycoside compound in combination with at least one other skincare ingredient or active agent for improving the appearance and/or the texture of the skin;
compositions comprising said combinations; or alternatively, a cosmetic or dermatological assembly comprising at least two separate compositions respectively containing, for one, the C-glycoside compound and, for the other, said cosmetic or dermatological ingredient and/or active agent; and
cosmetic methods using said combinations and/or said compositions.

### REFERENCE TO PRIOR APPLICATIONS

This application claims priority to U.S. provisional application 60/894,254 filed Mar. 12, 2007, incorporated herein by reference.

The invention relates, in particular, to:
novel uses of a C-glycoside compound;
the use, in a cosmetic composition in particular, of a C-glycoside compound in combination with at least one other skincare ingredient or active agent to improve the appearance of keratinous substances, in particular the skin;
compositions comprising said combinations; and
cosmetic methods using said combinations and/or said compositions.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

The expression “to improve the appearance of keratinous substances” is understood to mean to improve the aesthetic appearance of said substances, in particular to reduce surface irregularities in said keratinous substances and/or to improve the texture and/or the mechanical properties of said keratinous substances.

The cosmetic uses according to the invention comprise especially the improvement of the aesthetic appearance of the skin and/or its appendages, in particular improvement in the surface appearance and/or texture of the skin.

The term “surface appearance” is understood to mean, in particular, the visual and/or tactile irregularities in the skin and/or in the scalp, in particular wrinkles and fine lines, expression lines, in particular on the forehead and in the space between the eyebrows, wrinkles and/or fine lines around the mouth, and/or slackening in the area around the lips, in particular in the top lip area (area located between the top lip and the nose), heterogeneity of the skin tone (liver spots, actinic lentigos), appearance and/or visibility of the pores, the papery appearance of the skin, defects in the skin microrelief such as chicken pox or acne scars, imperfections of greasy skin (shiny appearance, etc.).

The term “skin texture” is understood to mean, especially, slack, flabby, less firm, less elastic skin, skin that has sagged.

Ageing of the area around the lips, in particular of the top lip area, is characteristic of women and especially menopausal women, in connection with an age-related hormonal imbalance. This ageing is especially expressed by a lengthening of the top lip area (increase in its height), by the appearance of vertical fine lines, by a hollowing of the nasal groove and a reduction of the fat mass of the cheek.

Expression lines are produced by the effect of the stress exerted on the skin by the skin muscles that enable expressions. Depending on the shape of the face, the frequency of expressions and possible tics, they may appear from childhood. Age, and also certain environmental factors such as exposure to sunlight are not involved in creating them but may deepen them further and make them permanent. Expression lines are characterized by the presence of grooves in the area around the orifices formed by the nose (nasal grooves), the mouth (perioral lines and so-called bitterness lines) and the eyes (crow's feet wrinkles), around which the skin muscles are located, and also between the eyebrows (glabella or lion wrinkles) and on the forehead. In particular, it will be sought to prevent and/or smooth out wrinkles on the forehead and in the space between the eyebrows.

The appearance and/or visibility of the pores is a relatively recent problem, which it is desired to treat, in particular in people who have dilated pores irrespective of their origin: ethnic (e.g.: Asiatic population, Caucasian populations), excess of sebum, ageing, loss of firmness, slackening, stress, fatigue, unsuitable hygiene, climatic factors, etc.

This skin imperfection, in particular on the face and especially in the T zone (forehead, nose, cheeks, chin), in particular on the nose and cheeks may be accentuated by an increase in the width of the conical portion around the pore and/or a parakeratotic state of the stratum corneum in this conical portion, itself associated with excessive secretion of sebum and of unsaturated fatty acids. Agents that are active on the pores improve the appearance and/or visibility of the pores, in particular the tightening, and therefore the reduction of the size of the dilated pores, thus making them less visible; the grain of the skin is thus tightened and refined, to give the surface of the skin a smoother appearance and feel and a refined grain; the skin is more radiant and/or transparent.

The papery appearance of the skin is characterized by a change in the visual appearance of the skin and also in the behaviour of the skin to the touch. More specifically, the skin visually takes on the appearance of cigarette paper giving it an appearance similar to that of a sheet of papyrus. Moreover, when it is gently pinched between the thumb and index finger the skin forms numerous fine sharp folds having the appearance of crumpled paper. Finally, the feel of the skin shows that its surface parts appear to be floating on the deeper parts, giving the skin, in the very advanced stage of the papery appearance, the appearance of creased paper. The papery appearance of the skin can be seen on the face and more characteristically still on the back of the hands of the elderly.

The dermatological uses according to the invention include the treatment of skin disorders linked to a change in the skin desquamation and/or pigmentation.

Desquamation disorders are understood, in particular, to include: xerosis, acne, hyperkeratoses, psoriasis, atopy and ichtyosis.

As pigmentation disorders, mention may especially be made of: melasma of the forearms, idiopathic melasmas, hyperpigmentations associated with pregnancy or with oestrogen-progestogen contraception, PUVA lentigines, accidental hyperpigmentation, hyperpigmentation due to leucoderma, and vitiligo.

The combination of a C-glycoside compound and another skincare ingredient/active agent according to the invention can be formulated in one and the same composition, or in two separate compositions, possibly being applied to the skin either simultaneously or successively or after a time delay.

The compositions according to the invention may be products for making up or caring for keratinous substances, in particular the skin.

More specifically, the makeup products may be of the following type: foundations, face powders or eye shadows, concealers, blusher, or else a body makeup or skin colouring product.

The skincare products may be, for example, a composition for protecting, treating or caring for the face, for the hands, for the feet, for large anatomic wrinkles or for the body (for example, day creams, night cream, makeup remover cream, anti-sun composition, body milks for skin protection or care, after-sun milks, skincare lotion, gel or foam, artificial tanning composition); an aftershave composition.

### I—C-Glycoside Compounds

The C-glycoside compounds used in the invention include, in particular, those described in document WO 02/051828, incorporated herein by reference.

C-glycoside compounds suitable for the invention include compounds of general formula (I) below:

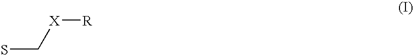

in which:
R represents:
   a saturated C₁ to C₂₀, in particular C₁ to C₁₀, or unsaturated C₂ to C₂₀, in particular C₂ to C₁₀, linear alkyl radical, or a saturated or unsaturated C₃ to C₂₀, in particular C₃ to C₁₀, branched or cyclic alkyl radical; or
   a saturated C₁ to C₂₀, in particular C₁ to C₁₀, or unsaturated C₂ to C₂₀, in particular C₂ to C₁₀, hydrofluoroalkyl or perfluoroalkyl radical, or a saturated or unsaturated C₃ to C₂₀, in particular C₃ to C₁₀, branched or cyclic hydrofluoroalkyl or perfluoroalkyl radical;
      the hydrocarbon chain making up said radicals optionally being interrupted by 1, 2, 3 or more heteroatoms chosen from:
oxygen;
sulphur;
nitrogen; and
silicon,
   and which may optionally be substituted by at least one radical chosen from:
—OR₄;
—SR₄;
—NR₄R₅;
—COOR₄;
—CONHR₄;
—CN;
a halogen atom;
a C₁ to C₆ hydrofluoroalkyl or perfluoroalkyl radical;
a C₃ to C₈ cycloalkyl radical; and/or
   with R₄ and R₅ possibly representing, independently of one another, a hydrogen atom, or a saturated C₁ to C₃₀, especially C₁ to C₁₂, or unsaturated C₂ to C₃₀, especially C₂ to C₁₂, linear alkyl, perfluoroalkyl or hydrofluoroalkyl radical, or a saturated or unsaturated C₃ to C₃₀, especially C₃ to C₁₂, branched or cyclic alkyl, perfluoroalkyl or hydrofluoroalkyl, or a C₆ to C₁₀ aryl radical,
X represents a radical chosen from the groups:

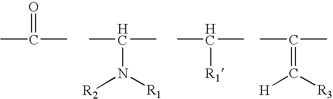

with R₁, R₂ and R₃ representing, independently of one another, a hydrogen atom, or a radical R, with R as defined previously, and R₁′ represents a hydrogen atom, an —OH group or a radical R as defined previously, R₁ possibly also denoting a C₆ to C₁₀ aryl radical;
S represents a monosaccharide or a polysaccharide composed of up to 20 sugar units, in particular up to 6 sugar units, in the form of pyranose and/or furanose and of L and/or D series, said monosaccharide or polysaccharide possibly being substituted by a compulsorily free hydroxyl group, and optionally one or more optionally protected amine functional groups; and
the S—CH₂—X linkage represents an anomeric C-type linkage, which may be α or β,
   and also their cosmetically acceptable salts, their solvates such as hydrates, alcoholates such as MeOH, EtOh, etc., and their isomers.

Within the scope of the present invention, the term “halogen” is understood to mean chlorine, fluorine, bromine or iodine.

The term “aryl” denotes an aromatic ring such as a phenyl ring, optionally substituted by one or more C₁-C₄ alkyl radicals.

The term “C₃ to C₈ cycloalkyl” denotes an aliphatic ring having from 3 to 8 carbon atoms, including for example cyclopropyl, cyclopentyl and cyclohexyl rings.

Among the alkyl groups that are suitable for use in the invention, mention may especially be made of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, a C-glycoside compound corresponding to the formula (I) may be used, for which S may represent a monosaccharide or a polysaccharide containing up to 6 sugar units, in the form of pyranose and/or furanose and of L and/or D series, said monosaccharide or polysaccharide having at least one compulsorily free hydroxyl functional group and/or optionally one or more compulsorily protected amine functional groups, X and R furthermore retaining all the previously given definitions.

Advantageously, a monosaccharide of the invention may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine, N-acetyl-D-galactosamine and advantageously denotes D-glucose, D-xylose, N-acetyl-D-glucosamine or L-fucose, and in particular, D-xylose.

More particularly, a polysaccharide of the invention containing up to 6 sugar units may be chosen from D-maltose, D-lactose, D-cellobiose, D-maltotriose, a dissacharide combining a uronic acid chosen from D-iduronic acid or D-glucuronic acid with a hexosamine chosen from D-galactosamine, D-glucosamine, N-acetyl-D-galactosamine, N-acetyl-D-glucosamine, an oligosaccharide containing at least one xylose which may advantageously be chosen from xylobiose, methyl-β-xylobioside, xylotriose, xylotetraose, xylopentaose and xylohexaose and especially the xylobiose that is composed of two xylose molecules joined via a 1-4 linkage.

More particularly, S may represent a monosaccharide chosen from D-glucose, D-xylose, L-fucose, D-galactose, D-maltose and especially D-xylose.

According to another embodiment of the invention, C-glycoside compounds corresponding to the formula (I) may be used, for which X represents a group chosen from —CO—, —CH(OH)—, —CH(NR₁R₂)—, —CH(R)—, in particular —CO—, —CH(OH)—, —CH(NH₂)—, —CH(NHCH₂CH₂CH₂OH)—, —CH(NHPh)-, —CH(CH₃)—, and more particularly a —CO—, —CH(OH)—, —CH(NH₂)— group, and preferentially a —CH(OH)— group, S and R furthermore retaining all the previously given definitions.

According to another embodiment of the invention, a C-glycoside compound corresponding to the formula (I) may be used, for which R represents a saturated C₁ to C₂₀, in particular C₁ to C₁₀, or unsaturated C₂ to C₂₀, in particular C₂ to C₁₀, linear alkyl radical, or a saturated or unsaturated C₃ to C₂₀, in particular C₃ to C₁₀, branched or cyclic alkyl radical, which is optionally substituted as described previously, S and R furthermore retaining all the previously given definitions. Preferably, R denotes a C₁-C₄, especially C₁-C₃, linear radical, optionally substituted by —OH, —COOH or —COOR″₂, R″₂ being a saturated C₁-C₄ alkyl radical, especially an ethyl radical. Preferentially R denotes an unsubstituted C₁-C₄, especially C₁-C₂ linear alkyl radical, in particular an ethyl radical.

Among the C-glycoside compounds of formula (I), preferably those for which the following applies are used:
R represents a saturated C₁ to C₂₀, in particular C₁ to C₁₀, or unsaturated C₂ to C₂₀, in particular C₂ to C₁₀, linear alkyl radical, or a saturated or unsaturated C₃ to C₂₀, in particular C₃ to C₁₀, branched or cyclic alkyl radical, which is optionally substituted as described previously;
S represents a monosaccharide as described previously; and
X represents —CO—, —CH(OH)—, —CH(NR₁R₂)—, —CH(R)— as described previously.

Preferably, a C-glycoside compound of formula (I) is used for which:
R denotes a C₁-C₄, especially C₁-C₃, linear radical, optionally substituted by —OH, —COOH or —COOK″₂, R″₂ being a saturated C₁-C₄ alkyl radical, especially an ethyl radical;
S represents a monosaccharide as described previously; and
X represents a group chosen from —CO—, —CH(OH)—, —CH(NH₂)—, —CH(NHCH₂CH₂CH₂OH)—, —CH(NHPh)-, —CH(CH₃)—, and more particularly a —CO—, —CH(OH)—, or —CH(NH₂)— group, and preferentially a —CH(OH)— group.

Preferentially, a C-glycoside compound of formula (I) is used for which:
R denotes an unsubstituted C₁-C₄, especially C₁-C₂, linear alkyl radical, in particular an ethyl radical;
S represents a monosaccharide as described previously; especially D-glucose, D-xylose, N-acetyl-D-glucosamine or L-fucose, and in particular D-xylose; and
X represents a group chosen from —CO—, —CH(OH)—, —CH(NH₂)—, and preferentially a —CH(OH)— group.

Acceptable salts for the non-therapeutic use of the compounds described in the present invention include the conventional non-toxic salts of said compounds, such as those formed from organic or inorganic acids. By way of example, mention may be made of the salts of mineral acids, such as sulphuric acid, hydrochloric acid, hydrobromic acid, hydroiodic acid, phosphoric acid, and boric acid. Mention may also be made of the salts of organic acids, which may comprise one or more carboxylic, sulphonic or phosphonic acid groups. They may be linear, branched or cyclic aliphatic acids or else aromatic acids. These acids may comprise, in addition, one or more heteroatoms chosen from O and N, for example in the form of hydroxyl groups. Mention may especially be made of propionic acid, acetic acid, terephthalic acid, citric acid and tartric acid.

When the compound of formula (I) comprises an acid group, neutralization of the acid group or groups may be carried out by a mineral base, such as LiOH, NaOH, KOH, Ca(OH)₂, NH₄OH, Mg(OH)₂ or Zn(OH)₂; or by an organic base such as a primary, secondary or tertiary alkylamine, for example triethylamine or butylamine. This primary, secondary or tertiary alkylamine may comprise one or more nitrogen and/or oxygen atoms and may therefore comprise, for example, one or more alcohol functional groups; mention may especially be made of 2-amino-2-methyl-propanol, triethanolamine, dimethylamino-2-propanol and 2-amino-2-(hydroxy-methyl)-1,3-propanediol. Mention may also be made of lysine or 3-(dimethylamino)propylamine.

Acceptable solvates for the compounds described in the present invention comprise conventional solvates such as those formed during the last step of preparing said compounds due to the presence of solvents. By way of example, mention may be made of the solvates due to the presence of water or linear or branched alcohols such as ethanol or isopropanol.

Among the C-glycoside compounds of formula (I), used according to the invention, the following are most particularly considered:
1. C-β-D-xylopyranoside-n-propan-2-one;
2. C-α-D-xylopyranoside-n-propan-2-one;
3. 1-[2-(3-hydroxypropylamino)propyl]-C-β-D-xylo-pyranose;
4. 1-[2-(3-hydroxypropylamino)propyl]-C-α-D-xylo-pyranose;
5. C-β-D-xylopyranoside-2-hydroxypropane;
6. C-α-D-xylopyranoside-2-hydroxypropane;
7. C-β-D-xylopyranoside-2-aminopropane;
8. C-α-D-xylopyranoside-2-aminopropane;
9. C-β-D-xylopyranoside-2-(phenylamino)propane;
10. C-α-D-xylopyranoside-2-(phenylamino)propane;
11. Ethyl ester of 3-methyl-4-(C-β-D-xylopyranoside)-butyric acid;
12. Ethyl ester of 3-methyl-4-(C-α-D-xylopyranoside)-butyric acid;
13. 6-(C-β-D-xylopyranoside)-5-ketohexanoic acid;
14. 6-(C-α-D-xylopyranoside)-5-ketohexanoic acid;
15. 6-(C-β-D-xylopyranoside)-5-hydroxyhexanoic acid;
16. 6-(C-α-D-xylopyranoside)-5-hydroxyhexanoic acid;
17. 6-(C-β-D-xylopyranoside)-5-aminohexanoic acid;
18. 6-(C-α-D-xylopyranoside)-5-aminohexanoic acid;
19. 6-(C-β-D-xylopyranoside)-5-(phenylamino)hexanoic acid;
20. 6-(C-α-D-xylopyranoside)-5-(phenylamino)hexanoic acid;
21. 1-(C-β-D-xylopyranoside)hexane-2,6-diol;
22. 1-(C-α-D-xylopyranoside)hexane-2,6-diol;
23. 5-(C-β-D-xylopyranoside)-4-ketopentanoic acid;
24. 5-(C-α-D-xylopyranoside)-4-ketopentanoic acid;
25. 5-(C-β-D-xylopyranoside)-4-hydroxypentanoic acid;
26. 5-(C-α-D-xylopyranoside)-4-hydroxypentanoic acid;
27. 5-(C-β-D-xylopyranoside)-4-aminopentanoic acid;
28. 5-(C-α-D-xylopyranoside)-4-aminopentanoic acid;
29. 5-(C-β-D-xylopyranoside)-4-(phenylamino)pentanoic acid;
30. 5-(C-α-D-xylopyranoside)-4-(phenylamino)pentanoic acid;
31. 1-(C-β-D-xylopyranoside)pentane-2,5-diol;
32. 1-(C-α-D-xylopyranoside)pentane-2,5-diol;
33. 1-(C-β-D-fucopyranoside)propan-2-one;
34. 1-(C-α-D-fucopyranoside)propan-2-one;
35. 1-(C-β-L-fucopyranoside)propan-2-one;
36. 1-(C-α-L-fucopyranoside)propan-2-one;
37. 1-(C-β-D-fucopyranoside)-2-hydroxypropane;
38. 1-(C-α-D-fucopyranoside)-2-hydroxypropane;
39. 1-(C-β-L-fucopyranoside)-2-hydroxypropane;
40. 1-(C-α-L-fucopyranoside)-2-hydroxypropane;
41. 1-(C-β-D-fucopyranoside)-2-aminopropane;
42. 1-(C-α-D-fucopyranoside)-2-aminopropane;
43. 1-(C-β-L-fucopyranoside)-2-aminopropane;
44. 1-(C-α-L-fucopyranoside)-2-aminopropane;
45. 1-(C-β-D-fucopyranoside)-2-(phenylamino)propane;
46. 1-(C-α-D-fucopyranoside)-2-(phenylamino)propane;
47. 1-(C-β-L-fucopyranoside)-2-(phenylamino)propane;
48. 1-(C-α-L-fucopyranoside)-2-(phenylamino)propane;
49. Ethyl ester of 3-methyl-4-(C-β-D-fucopyranoside)-butyric acid;
50. Ethyl ester of 3-methyl-4-(C-α-D-fucopyranoside)-butyric acid;
51. Ethyl ester of 3-methyl-4-(C-β-L-fucopyranoside)-butyric acid;
52. Ethyl ester of 3-methyl-4-(C-α-L-fucopyranoside)-butyric acid;
53. 6-(C-β-D-fucopyranoside)-5-ketohexanoic acid;
54. 6-(C-α-D-fucopyranoside)-5-ketohexanoic acid;
55. 6-(C-β-L-fucopyranoside)-5-ketohexanoic acid;
56. 6-(C-α-L-fucopyranoside)-5-ketohexanoic acid;
57. 6-(C-β-D-fucopyranoside)-5-hydroxyhexanoic acid;
58. 6-(C-α-D-fucopyranoside)-5-hydroxyhexanoic acid;
59. 6-(C-β-L-fucopyranoside)-5-hydroxyhexanoic acid;
60. 6-(C-α-L-fucopyranoside)-5-hydroxyhexanoic acid;
61. 6-(C-β-D-fucopyranoside)-5-aminohexanoic acid;
62. 6-(C-α-D-fucopyranoside)-5-aminohexanoic acid;
63. 6-(C-β-L-fucopyranoside)-5-aminohexanoic acid;
64. 6-(C-α-L-fucopyranoside)-5-aminohexanoic acid;
65. 1-(C-β-D-fucopyranoside)hexane-2,6-diol;
66. 1-(C-α-D-fucopyranoside)hexane-2,6-diol;
67. 1-(C-β-L-fucopyranoside)hexane-2,6-diol;
68. 1-(C-α-L-fucopyranoside)hexane-2,6-diol;
69. 5-(C-β-D-fucopyranoside)-4-ketopentanoic acid;
70. 5-(C-α-D-fucopyranoside)-4-ketopentanoic acid;
71. 5-(C-β-L-fucopyranoside)hexane-2,6-diol)-4-keto-pentanoic acid;
72. 5-(C-α-L-fucopyranoside)hexane-2,6-diol)-4-keto-pentanoic acid;
73. 5-(C-β-D-fucopyranoside)-4-hydroxypentanoic acid;
74. 5-(C-α-D-fucopyranoside)-4-hydroxypentanoic acid;
75. 5-(C-β-L-fucopyranoside)-4-hydroxypentanoic acid;
76. 5-(C-α-L-fucopyranoside)-4-hydroxypentanoic acid;
77. 5-(C-β-D-fucopyranoside)-4-aminopentanoic acid;
78. 5-(C-α-D-fucopyranoside)-4-aminopentanoic acid;
79. 5-(C-β-L-fucopyranoside)-4-aminopentanoic acid;
80. 5-(C-α-L-fucopyranoside)-4-aminopentanoic acid;
81. 1-(C-β-D-fucopyranoside)pentane-2,5-diol;
82. 1-(C-α-D-fucopyranoside)pentane-2,5-diol;
83. 1-(C-β-L-fucopyranoside)pentane-2,5-diol;
84. 1-(C-α-L-fucopyranoside)pentane-2,5-diol;
85. 1-(C-β-D-glucopyranosyl)-2-hydroxypropane;
86. 1-(C-α-D-glucopyranosyl)-2-hydroxypropane;
87. 1-(C-β-D-glucopyranosyl)-2-aminopropane;
88. 1-(C-α-D-glucopyranosyl)-2-aminopropane;
89. 1-(C-β-D-glucopyranosyl)-2-(phenylamino)propane;
90. 1-(C-α-D-glucopyranosyl)-2-(phenylamino)propane;
91. Ethyl ester of 3-methyl-4-(C-β-D-glucopyranosyl)-butyric acid;
92. Ethyl ester of 3-methyl-4-(C-α-D-glucopyranosyl)-butyric acid;
93. 6-(C-β-D-glucopyranosyl)-5-ketohexanoic acid;
94. 6-(C-α-D-glucopyranosyl)-5-ketohexanoic acid;
95. 6-(C-β-D-glucopyranosyl)-5-hydroxyhexanoic acid;
96. 6-(C-α-D-glucopyranosyl)-5-hydroxyhexanoic acid;
97. 6-(C-β-D-glucopyranosyl)-5-aminohexanoic acid;
98. 6-(C-α-D-glucopyranosyl)-5-aminohexanoic acid;
99. 6-(C-β-D-glucopyranosyl)-5-(phenylamino)hexanoic acid;
100. 6-(C-α-D-glucopyranosyl)-5-(phenylamino)hexanoic acid;
101. 1-(C-β-D-glucopyranosyl)hexane-2,6-diol;
102. 1-(C-α-D-glucopyranosyl)hexane-2,6-diol;
103. 6-(C-β-D-glucopyranosyl)-5-ketopentanoic acid;
104. 6-(C-α-D-glucopyranosyl)-5-ketopentanoic acid;
105. 6-(C-β-D-glucopyranosyl)-5-hydroxypentanoic acid;
106. 6-(C-α-D-glucopyranosyl)-5-hydroxypentanoic acid;
107. 6-(C-β-D-glucopyranosyl)-5-aminopentanoic acid;
108. 6-(C-α-D-glucopyranosyl)-5-aminopentanoic acid;
109. 6-(C-β-D-glucopyranosyl)-5-(phenylamino)pentanoic acid;
110. 6-(C-α-D-glucopyranosyl)-5-(phenylamino)pentanoic acid;
111. 1-(C-β-D-glucopyranosyl)pentane-2,5-diol;
112. 1-(C-α-D-glucopyranosyl)pentane-2,5-diol;
113. 1-(C-β-D-galactopyranosyl)-2-hydroxypropane;
114. 1-(C-α-D-galactopyranosyl)-2-hydroxypropane;
115. 1-(C-β-D-galactopyranosyl)-2-aminopropane;
116. 1-(C-α-D-galactopyranosyl)-2-aminopropane;
117. 1-(C-β-D-galactopyranosyl)-2-(phenylamino)propane;
118. 1-(C-α-D-galactopyranosyl)-2-(phenylamino)propane;
119. Ethyl ester of 3-methyl-4-(β-D-galactopyranosyl)-butyric acid;
120. Ethyl ester of 3-methyl-4-(α-D-galactopyranosyl)-butyric acid;
121. 6-(C-β-D-galactopyranosyl)-5-ketohexanoic acid;
122. 6-(C-α-D-galactopyranosyl)-5-ketohexanoic acid;
123. 6-(C-β-D-galactopyranosyl)-5-hydroxyhexanoic acid;
124. 6-(C-α-D-galactopyranosyl)-5-hydroxyhexanoic acid;
125. 6-(C-β-D-galactopyranosyl)-5-aminohexanoic acid;
126. 6-(C-α-D-galactopyranosyl)-5-aminohexanoic acid;
127. 6-(C-β-D-galactopyranosyl)-5-(phenylamino)hexanoic acid;
128. 6-(C-α-D-galactopyranosyl)-5-(phenylamino)hexanoic acid;
129. 1-(C-β-D-galactopyranosyl)hexane-2,6-diol;
130. 1-(C-α-D-galactopyranosyl)-hexane-2,6-diol;
131. 6-(C-β-D-galactopyranosyl)-5-ketopentanoic acid;
132. 6-(C-α-D-galactopyranosyl)-5-ketopentanoic acid;
133. 6-(C-β-D-galactopyranosyl)-5-hydroxypentanoic acid;
134. 6-(C-α-D-galactopyranosyl)-5-hydroxypentanoic acid;
135. 6-(C-β-D-galactopyranosyl)-5-aminopentanoic acid;
136. 6-(C-α-D-galactopyranosyl)-5-aminopentanoic acid;
137. 6-(C-β-D-galactopyranosyl)-5-(phenylamino)-pentanoic acid;
138. 6-(C-α-D-galactopyranosyl)-5-(phenylamino)-pentanoic acid;
139. 1-(C-β-D-galactopyranosyl)pentane-2,6-diol;
140. 1-(C-α-D-galactopyranosyl)pentane-2,6-diol;
141. 1-(C-β-D-fucofuranosyl)propan-2-one;
142. 1-(C-α-D-fucofuranosyl)propan-2-one;
143. 1-(C-β-L-fucofuranosyl)propan-2-one;
144. 1-(C-α-L-fucofuranosyl)propan-2-one;
145. 3′-(acetamido-C-β-D-glucopyranosyl)propan-2′-one;
146. 3′-(acetamido-C-α-D-glucopyranosyl)propan-2′-one;
147. 1-(acetamido-C-β-D-glucopyranosyl)-2-hydroxyl-propane;
148. 1-(acetamido-C-β-D-glucopyranosyl)-2-aminopropane;
149. 1-(acetamido-C-β-D-glucopyranosyl)-2-(phenylamino)propane;
150. 1-(acetamido-C-α-D-glucopyranosyl)-2-(phenylamino)propane;
151. Ethyl ester of 3-methyl-4-(acetamido-C-β-D-gluco-pyranosyl)butyric acid;
152. Ethyl ester of 3-methyl-4-(acetamido-C-α-D-gluco-pyranosyl)butyric acid;
153. 6-(acetamido-C-β-D-glucopyranosyl)-5-ketohexanoic acid;
154. 6-(acetamido-C-α-D-glucopyranosyl)-5-ketohexanoic acid;
155. 6-(acetamido-C-β-D-glucopyranosyl)-5-hydroxy-hexanoic acid;
156. 6-(acetamido-C-α-D-glucopyranosyl)-5-hydroxy-hexanoic acid;
157. 6-(acetamido-C-β-D-glucopyranosyl)-5-aminohexanoic acid;
158. 6-(acetamido-C-α-D-glucopyranosyl)-5-aminohexanoic acid;
159. 6-(acetamido-C-β-D-glucopyranosyl)-5-(phenylamino)hexanoic acid;
160. 6-(acetamido-C-α-D-glucopyranosyl)-5-(phenylamino)hexanoic acid;
161. 1-(acetamido-C-β-D-glucopyranosyl)hexane-2,6-diol;
162. 1-(acetamido-C-α-D-glucopyranosyl)hexane-2,6-diol;
163. 6-(acetamido-C-β-D-glucopyranosyl)-5-ketopentanoic acid;
164. 6-(acetamido-C-α-D-glucopyranosyl)-5-ketopentanoic acid;
165. 6-(acetamido-C-β-D-glucopyranosyl)-5-hydroxypentanoic acid;
166. 6-(acetamido-C-α-D-glucopyranosyl)-5-hydroxypentanoic acid;
167. 6-(acetamido-C-β-D-glucopyranosyl)-5-aminopentanoic acid;
168. 6-(acetamido-C-α-D-glucopyranosyl)-5-aminopentanoic acid;
169. 6-(acetamido-C-β-D-glucopyranosyl)-5-(phenylamino)pentanoic acid;
170. 6-(acetamido-C-α-D-glucopyranosyl)-5-(phenylamino)pentanoic acid;
171. 1-(acetamido-C-β-D-glucopyranosyl)pentane-2,5-diol;
172. 1-(acetamido-C-α-D-glucopyranosyl)pentane-2,5-diol.

As a non-limiting illustration of the C-glycoside compounds that are more particularly suitable for the invention, mention may especially be made of the following compounds:
C-β-D-xylopyranoside-n-propan-2-one;
C-α-D-xylopyranoside-n-propan-2-one;
C-β-D-xylopyranoside-2-hydroxypropane;
C-α-D-xylopyranoside-2-hydroxypropane;
1-(C-β-D-fucopyranoside)propan-2-one;
1-(C-α-D-fucopyranoside)propan-2-one;
1-(C-β-L-fucopyranoside)propan-2-one;
1-(C-α-L-fucopyranoside)propan-2-one;
1-(C-β-D-fucopyranoside)-2-hydroxypropane;
1-(C-α-D-fucopyranoside)-2-hydroxypropane;
1-(C-β-L-fucopyranoside)-2-hydroxypropane;
1-(C-α-L-fucopyranoside)-2-hydroxypropane;
1-(C-β-D-glucopyranosyl)-2-hydroxypropane;
1-(C-α-D-glucopyranosyl)-2-hydroxypropane;
1-(C-β-D-galactopyranosyl)-2-hydroxypropane;
1-(C-α-D-galactopyranosyl)-2-hydroxypropane;
1-(C-β-D-fucofuranosyl)propan-2-one;
1-(C-α-D-fucofuranosyl)propan-2-one;
1-(C-β-L-fucofuranosyl)propan-2-one;
1-(C-α-L-fucofuranosyl)propan-2-one;
(C-β-D-maltopyranoside-n-propan-2-one;
(C-α-D-maltopyranoside-n-propan-2-one;
(C-β-D-maltopyranoside-2-hydroxypropane; and
(C-α-D-maltopyranoside-2-hydroxpropane, isomers and blends thereof.

According to one embodiment, C-β-D-xylopyranoside-2-hydroxypropane or C-α-D-xylopyranoside-2-hydroxypropane, and in a highly preferred embodiment C-β-D-xylopyranoside-2-hydroxypropane, may advantageously be used for preparing a composition according to the invention.

According to one particular embodiment, the C-glycoside compound is C-α-D-xylopyranoside-2-hydroxypropane in the form of a 30 wt % solution of active material in a water/propylene glycol (60/40 wt %) mixture such as the product manufactured by Chimex under the trade name “MEXORYL SBB®”.

Of course, according to the invention, a C-glycoside compound corresponding to the formula (I) may be used alone or as a blend with other C-glycoside compounds and in any proportion.

A C-glycoside compound that is suitable for the invention may especially be obtained by the synthesis method described in document WO 02/051828, incorporated herein by reference.

The amount of C-glycoside compound used in a composition according to the invention depends on the desired cosmetic or therapeutic effect, and may therefore vary over a wide range.

A person skilled in the art may easily, based on their general knowledge, determine the appropriate amounts.

A composition according to the invention may comprise a C-glycoside compound in an amount of around 0.0001% to around 25% by weight of active material relative to the total weight of the composition, and in particular from around 0.001% to around 10% by weight of active material, and more particularly from around 0.05% to around 5% by weight of active material of C-glycoside compound relative to the total weight of the composition.

II—Cosmetic or Dermatological Ingredients and/or Active Agents

The term “ingredient” is especially understood, according to the invention, to mean an ingredient favouring the solubilization, stabilization and/or activity of said C-glycoside compound.

The term “ingredient favouring the stabilization of said previously described C-glycoside compounds” is understood according to the invention to mean, in particular, an ingredient making it possible to (i) either stabilize said C-glycoside compound, or (ii) stabilize the physiologically acceptable medium in which said C-glycoside compound is present.

### A—Ingredients Favouring the Solubilization of Said C-Glycoside Compounds

As ingredient favouring the solubilization of the (hydrophilic) C-glycoside compounds, aqueous or aqueous-alcoholic solvents can be used.

As solvents, water and linear or branched alcohols comprising from 1 to 8 carbon atoms, and in particular to 6 carbon atoms, such as ethanol, isopropanol, propanol and butanol; polyethylene glycols having 6 to ethylene oxide units; polyols such as propylene glycol, isoprene glycol, butylene glycol, glycerol and sorbitol will be used.

When the physiologically acceptable medium is an aqueous medium, it generally has a pH compatible with the skin, preferably ranging from 3 to 9 and better still from 3.5 to 7.5.

### B—Ingredients Favouring the Stabilization of Said C-Glycoside Compounds

The term “ingredient favouring the stabilization of said C-glycoside compounds” is understood according to the invention to mean, in particular, an ingredient making it possible to (i) either stabilize said C-glycoside compound, or (ii) stabilize the physiologically acceptable medium in which said C-glycoside compound is present.

In particular, in the case (ii), the ingredient favouring the stabilization of said C-glycoside compounds may especially be present in a particular galenic form that contributes to the stabilization of said C-glycoside compound in said physiologically acceptable medium.

As ingredients for stabilizing said C-glycoside compounds that can be used in the compositions of the invention, mention may especially be made of:
(a) block polymers and/or copolymers;
(b) ionic or non-ionic type amphiphilic lipids present in the form of vesicles in dispersion;
(c) constituent polymers of nanoparticles, in particular of nanospheres or nanocapsules;
(d) constituent polymers of microparticles;
(e) polymers and/or surfactants forming nanoemulsions;
(f) polymers in the form of thin films;
(g) emulsifiers based on polyolefins having a polar part, the composition being in the form of a water-in-oil emulsion; and
(h) amphiphilic polymers comprising 2-acrylamido-2-methylpropanesulphonic acid (AMPS) units.
(a) Block polymers and/or copolymers

It is known how to encapsulate active agents into micelles of block copolymers, for example polyethylene oxide/propylene oxide diblock or triblock copolymers.

Advantageously, an amphiphilic block copolymer comprising at least one non-ionic hydrophilic polymer block and at least one particular hydrophobic polymer block will be used in the composition of the invention.

These amphiphilic block copolymers are especially described in Patent Application EP 1 555 984, incorporated here by reference.

The molecular weight of the block copolymer is generally between 1000 and 100 000.

In particular, the weight ratio of the ionic or non-ionic hydrophilic polymer block(s) to the hydrophobic polymer block(s) is between 1/100 and 50/1.

The hydrophobic polymer block is especially chosen from:
styrene and its compounds, such as 4-butylstyrene;
alkylene oxides comprising more than 4 carbon atoms, and preferably from 4 to 6 carbon atoms;
hydrophobic vinyl monomers of formula (A) below:

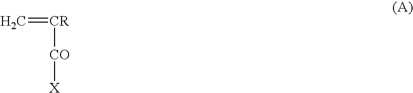

in which:
R is chosen from H or —CH₃,
X is chosen from —OR′ type alkyl oxides where R′ is a saturated or unsaturated, linear or branched, hydrocarbon-based radical having from 1 to 22 carbon atoms.

Preferably, the hydrophobic polymer block is obtained from one or more hydrophobic monomers chosen from methyl methacrylate, ethyl methacrylate, n-butyl (meth)acrylate, tert-butyl (meth)acrylate and cyclohexyl acrylate.

In particular, the hydrophobic polymer block is chosen from polystyrene, poly(tert-butylstyrene), polymethyl methacrylate, polyethyl acrylate, polybutyl methacrylate, and C₃-C₆ polyalkylene oxides.

Preferably, the non-ionic hydrophilic polymer block is chosen from polyethylene oxides.

Preferably, the block copolymer is chosen from the following block copolymers:
polystyrene/polyoxyethylene;
polymethyl methacrylate/polyoxyethylene;
polybutyl methacrylate/polyoxyethylene; and
polyoxyethylene/polyoxybutylene/polyoxyethylene

### (b) Ionic or Non-Ionic Type Amphiphilic Lipids

According to another embodiment of the invention, the C-glycoside compounds are combined with ionic or non-ionic amphiphilic lipids present in the form of ionic (e.g. liposomes) and/or non-ionic (e.g. niosomes) vesicles in dispersion in the physiologically acceptable medium of the composition, in particular in an aqueous dispersion.

The presence of these vesicles contributes to the stabilization of said C-glycoside compounds in the physiologically acceptable medium of the composition.

These lipid vesicles may have an aqueous core or an oily core.

Preferentially, lipid vesicles having an oily core will be used.

The term “vesicle” is understood, according to the invention, to mean any particulate structure comprising, on the one hand, a membrane or “lipid phase” formed from one or more concentric sheets, these sheets comprising one or more bimolecular layers based on ionic or non-ionic amphiphilic lipids and, on the other hand, an aqueous or oily phase encapsulated by this lipid phase. According to the meaning of the invention, the liposomes and niosomes form, in particular, such vesicles.

Niosomes are vesicles prepared from non-ionic amphiphilic lipids. Reference may especially be made to the description of Patent FR 8 907 947, incorporated into the present invention by reference.

As non-ionic amphiphilic lipid, mention may especially be made of optionally oxyethylenated alkyl or polyalkyl esters, and optionally oxyethylenated polyol ethers, having a melting point of at least 40° C.

Liposomes are vesicles prepared from ionic amphiphilic lipids. These vesicles are particles formed from a membrane composed of one or more concentric sheets, these sheets comprising one or more bimolecular layers of amphiphilic lipids encapsulating an aqueous or oily phase. The aqueous phase may contain water-soluble active substances and the bimolecular layers of amphiphilic lipids may contain lipophilic active substances. These vesicles generally have an average diameter between 10 and 5000 nanometres.

The ionic amphiphilic lipids may be anionic amphiphilic lipids or cationic amphiphilic lipids.

As examples of anionic amphiphilic lipids, mention may especially be made of:
preferably, neutralized anionic lipids, chosen from the alkali-metal salts of dicetyl phosphate and of dimyristyl phosphate, in particular the sodium and potassium salts, the alkali-metal salts of phosphatidic acid, in particular the sodium salt, the alkali-metal salts of cholesterol sulphate, in particular the sodium salt, the alkali-metal salts of cholesterol phosphate, in particular the sodium salt, the salts of lipoamino acids such as monosodium or disodium acylglutamates, more particularly the disodium salt of N-stearoyl-L-glutamic acid sold under the name ACYLGLUTAMATE HS21 by Ajinomoto;
amphoteric lipids, in particular pure soybean phosphatidylethanolamine; and alkylsulphonic compounds.

As cationic amphiphilic lipids, quaternary ammonium salts, fatty amines and salts thereof may especially be used.

It is possible to advantageously use “double liposome” compositions for simultaneously treating superficial and deep skin layers, comprising a first dispersion of lipid vesicles capable of penetrating into the deep skin layers and containing at least one active agent capable of treating these deep layers and a second dispersion of lipid vesicles capable of penetrating into the superficial skin layers and containing at least one active agent capable of treating these superficial layers. Such a system is described in Patent EP 0 661 035, incorporated here by reference.

Oleosomes are oily globules provided with a lamellar liquid crystal coating that are dispersed in an aqueous phase, having an average diameter that is generally less than 500 nanometres.

As an example of a formulation in oleosomes, reference may especially be made to Patent EP 0 641 557, incorporated into the present invention by reference.

### (c) Constituent Polymers of Nanoparticles

According to one alternative, it will be possible to combine said C-glycoside compounds with small-sized particles, in particular known as nanoparticles.

They could be solid particles formed from the combination of said C-glycoside compounds with at least one polymer.

This polymer contributes towards stabilizing said C-glycoside compound in the physiologically acceptable medium of the composition.

The term “nanoparticles” mainly encompasses two different systems: “nanospheres” formed from a polymer matrix in which said C-glycoside compound is absorbed and/or adsorbed and/or mixed, and also “nanocapsules” having a core-shell type structure, that is to say a structure composed of a lipid core that is liquid at room temperature containing the C-glycoside compound in dissolved form, which core is encapsulated in a continuous protective shell that is insoluble in the medium.

Preferably nanocapsules will be used.

Reference may be made, for example, to the description of Patent Application EP 1 414 390, incorporated here by reference.

The nanospheres generally have an average size between 50 and 500 nm.

The nanocapsules are generally small-sized so as to obtain optimal bioavailability of the C-glycoside compounds.

Preferentially, the size of these nanocapsules is between 10 nm and 1000 nm and more particularly between 30 nm and 500 nm.

It will especially be possible to use polymers in the form of nanocapsules as described in Patent Application EP 0 274 961, the nanocapsules provided with a lamellar coating described in Application EP 0 780 115, the nanocapsules whose continuous polymer shell, which is insoluble in water, is formed from polyesters, as described in Applications EP 1 025 901, FR 2 787 730 and EP 1 034 839, or else the biodegradable nanocapsules described in Patent Application FR 2 659 554, or the non-biodegradable nanocapsules described in Patent Application WO 93/05753.

The nanocapsules made from biodegradable polymers penetrate into the skin and are degraded in the epidermis under the action of enzymes that are present therein, whereas the nanocapsules made from non-biodegradable polymers only penetrate into the superficial layers of the stratum corneum and are naturally eliminated during skin renewal.

As constituent polymers of nanocapsules that can be used in the compositions of the invention in combination with the C-glycoside compounds, mention may especially be made of poly-L-lactides and poly-DL-lactides and polycaprolactones, polyglycolides and copolymers thereof, polymers derived from the polymerization of an alkyl cyanoacrylate (the alkyl chain having from 2 to 6 carbon atoms); synthetic or natural water-dispersible anionic polymers; polyesters of the poly(alkylene adipate) type; dendritic polymers; vinyl chloride/vinyl acetate copolymers, copolymers of methacrylic acid and of the methyl ester of methacrylic acid, polyvinyl acetophthalate, cellulose acetophthalate, crosslinked polyvinyl pyrrolidone/vinyl acetate copolymers, polyethylene vinyl acetates, polyacrylonitriles, polyacrylamides, polyethylene glycols, polyamides, polyethylenes, polypropylenes and organopolysiloxanes.

Preferably, polycaprolactones will be used.

### (d) Constituent Polymers of Microparticles

According to one particular embodiment of the invention, especially advantageous for the care of greasy skin, the C-glycoside compounds will be combined with constituent polymers of microparticles. These polymers contribute towards stabilizing said C-glycoside compound in the physiologically acceptable medium of the composition.

The term “microparticles” especially encompasses “porous particles” and in particular “microspheres”.

The term “porous particles” is understood to denote particles having a structure comprising pores. This porous structure may, at least partially, make it possible to incorporate one or more active agents within said particles.

Reference may especially be made to Patent Application EP 1 637 124, incorporated here by reference.

The structure of the particles may be of the matrix type similar to a sponge. It may also be of the vesicle type, that is to say that the particle has an internal cavity delimited by a porous wall. The porosity associated with the size of the particles is characterized quantitatively by their specific surface area.

Porous particles will especially be used having a specific surface area measured according to the BET method greater than or equal to 1 m²/g. The BET (Brunauer-Emmet-Teller) method is a method that is well known to a person skilled in the art. It is especially described in “The Journal of the American Chemical Society”, Vol. 60, page 309, February 1938, and corresponds to the International Standard ISO 5794/1 (Annex D). The specific surface area measured according to the BET method corresponds to the total specific surface area, that is to say that it includes the surface formed by the pores.

According to one particular embodiment, the particles of the invention have a specific surface area measured by the BET method, ranging, in particular, from 2.5 to 100 m²/g.

The porous particles that can be used in the compositions of the invention are generally individual particles. The expression “individual particles” denotes particles that are not grouped together in the form of an aggregate or agglomerate. These particles have, in particular, a density greater than or equal to 0.15 g/cm³ and especially ranging from 0.2 to 5 g/cm³.

These particles preferably have a volume-average diameter less than or equal to 10 μm. Indeed, such particles can penetrate into the sebaceous follicle by application of a mechanical force. This mechanical force generally results from a massage which, besides the pressure that it exerts, generates a pump effect at the follicle. The particles thus gradually reach the follicle channel in which they are capable of absorbing sebum and, where appropriate, of releasing the active compound that they carry. The constituent material of the particles is then discharged thanks to the flow of sebum and/or the growth of the hair, thus enabling any possible undesirable reaction of the body towards this material to be prevented.

In particular, particles, especially spherical porous particles having a number-average size which may range from 0.1 to 50 μm, especially from 0.1 to 20 μm and most particularly from 0.5 to 10 μm, will be used.

As preferred porous particles, it is possible to use polyamide particles in particular nylon-6, nylon-6,6, nylon-12 or nylon-6,12 such as those sold by Atofina under their generic name ORGASOL.

This encapsulation system, which allows follicular targeting, is particularly advantageous in compositions intended for treating greasy skin.

(e) Polymers and/or Surfactants Forming Nanoemulsions

According to another embodiment of the invention, said C-glycoside compounds are combined in the composition with particular polymers and/or surfactants, the composition being in the form of a nanoemulsion.

These polymers and/or surfactants contribute towards stabilizing said C-glycoside compounds in the physiologically acceptable medium of the composition.

The nanoemulsions are generally oil-in-water emulsions whose oil globules have a very fine particle size distribution, that is to say a number-average size less than 100 nanometres (nm).

Reference may especially be made to the description of Patent EP 0 728 460, incorporated here by reference.

The nanoemulsions may be stabilized by a lamellar liquid crystal coating obtained by combining a hydrophilic surfactant with a lipophilic surfactant.

Advantageously, the particular polymers that make it possible to produce nanoemulsions may be chosen from:
anionic polymers having a hydrophobic chain, as described in Patent EP 116 005, incorporated here by reference; and/or
water-soluble non-ionic polymers as described in Patent EP 1 172 077, incorporated here by reference.

The particular surfactants that make it possible to produce nanoemulsions may, in particular, be a ternary surfactant system including a mixture of non-ionic surfactants and an ionic surfactant, as described in Patent EP 1 353 629, incorporated here by reference.

In particular, the anionic polymer having a hydrophobic chain comprises hydrophobic chains chosen from saturated or unsaturated, linear or branched hydrocarbon-based chains having from 6 to 30 carbon atoms, divalent cycloaliphatic groups and divalent aromatic groups, and preferably chosen from alkyl, arylalkyl, alkylaryl, alkylene, methylenedicyclohexyl, isophorone and phenylene chains.

In particular, the anionic polymer is chosen from copolymers of acrylic or methacrylic acid, copolymers of 2-acrylamido-2-methylpropanesulphonic acid and blends thereof.

Preferably, the anionic polymer is obtained by copolymerizing a monomer (a) chosen from α,β-ethylenically unsaturated carboxylic acids (monomer a′) and 2-acrylamido-2-methylpropanesulphonic acid (monomer a″), with a non-surfactant ethylenically unsaturated monomer (b) different from (a) and/or an ethylenically unsaturated monomer (c) derived from the reaction of an α,β-monoethylenically unsaturated acrylic monomer or a monoethylenically unsaturated isocyanate monomer with a monohydric non-ionic amphiphilic component or with a primary or secondary fatty amine.

Advantageously, the anionic polymer is an acrylic terpolymer obtained from (a) an α,β-ethylenically unsaturated carboxylic acid, (b) a non-surfactant ethylenically unsaturated monomer different from (a) and (c) a non-ionic urethane monomer that is the reaction product of a monohydric non-ionic amphiphilic compound with a monoethylenically unsaturated isocyanate.

For example, the anionic polymer is chosen from the acrylic acid/ethyl acrylate/alkyl acrylate terpolymer, the acrylates/steareth-20 methacrylate copolymer, the oxyethylenated (25 EO) (meth)acrylic acid/ethyl acrylate/behenyl methacrylate terpolymer, the oxyethylenated (20 EO) acrylic acid/monocetyl itaconate copolymer, the oxyethylenated (20 EO) acrylic acid/monostearyl itaconate copolymer, the acrylates/acrylate modified by polyoxyethylenated (25 EO) C₁₂-C₂₄ alcohols copolymer, the ethoxylated methacrylic acid/methyl acrylate/behenyl alcohol dimethyl-meta-isopropenyl benzylisocyanate terpolymer and blends thereof.

In particular, the water-soluble non-ionic polymer may be chosen from ethylene oxide homopolymers and copolymers; polyvinyl alcohols; vinylpyrrolidone homopolymers and copolymers; vinylcaprolactam homopolymers and copolymers; polyvinyl methyl ether homopolymers and copolymers; neutral acrylic homopolymers and copolymers; C₁-C₂ alkyl celluloses and their compounds; C₁-C₃ alkyl guar or C₁-C₃ hydroxyalkyl guar.

The ternary surfactant system may comprise in particular:

(a) a mixture of at least two non-ionic surfactants comprising at least one ethoxylated fatty ester having 8 to 100 units (especially 10 to 80 units, and best 40 units) of ethylene oxide and at least one sorbitan fatty acid ester; and
(b) at least one ionic surfactant chosen from the alkali-metal salts of cetyl phosphate and the alkali-metal salts of palmitoyl sarcosinate.

The ethoxylated fatty ester is preferably polyethylene glycol (40 EO) stearate and the sorbitan fatty acid ester is preferably sorbitan tristearate.

The ionic surfactant is especially chosen from potassium cetyl phosphate, sodium palmitoyl sarcosinate and mixtures thereof.

### (f) Polymers in the Form of Thin Films

According to another embodiment of the invention, said C-glycoside compounds are combined with at least one water-soluble or water-dispersible polymer in the form of a thin film.

The composition is therefore in the form of a thin film.

This water-soluble or water-dispersible polymer contributes towards stabilizing C-glycoside compounds in the physiologically acceptable medium of the composition.

The term “film” is understood in the present application to mean a thin solid that can be grasped. The term “thin” is understood to mean a solid having a maximum thickness of 1000 μm. This film generally has a sufficient size to be able to be easily handled by the user. It may be in the shape of a square, rectangle, disc or any other shape. Each film generally has a thickness from 10 μm to 1000 μm, preferably from 20 to 500 μm and best from 50 to 300 μm. It may have a surface area of 0.25 to 25 cm² and preferably from 2 to 10 cm².

These thin films generally contain less than 10% by weight of water, preferably less than 5% by weight relative to the total weight of the film, and more preferably, do not contain any water.

Such films are described in Patent Application EP 1 588 694, incorporated here by reference.

The thin film comprises a water-soluble or water-dispersible polymer which may be chosen from: (1) proteic-type polymers, such as wheat or soybean proteins; keratin, for example keratin hydrolysates and sulphonic keratins; casein; albumin; collagen; glutelin; glucagon; gluton; zein; gelatins and their compounds; (2) polymers deriving from chitin or chitosan, such as anionic, cationic, amphoteric or non-ionic chitin or chitosan polymers; (3) polysaccharide polymers such as, especially (i) cellulose polymers, such as hydroxyethyl cellulose, hydroxypropyl cellulose, hydroxypropyl methyl cellulose, methyl cellulose, ethyl hydroxyethyl cellulose, carboxymethyl cellulose, quaternary compounds of cellulose; and (ii) starches and their compounds; (4) acrylic polymers or copolymers such as polyacrylates, polymethacrylates and copolymers thereof; (5) vinyl polymers such as polyvinylpyrrolidones, methyl vinyl ether/maleic anhydride copolymers, vinyl acetate/crotonic acid copolymer, vinylpyrrolidone/vinyl acetate copolymers, vinylpyrrolidone/caprolactam copolymers and polyvinyl alcohols; (6) optionally modified polymers of natural origin, such as gum arabic, guar gum, xanthan compounds, karaya gum; alginates, carrageenans, ulvans and other algal colloids; glycoaminoglycans, hyaluronic acid and its compounds; shellac, gum sandarac, dammars, elemi gum, copals; deoxyribonucleic acid; mucopolysaccharides, such as hyaluronic acid, chondroitin sulphate; and blends of these polymers.

Mention may also be made, as water-soluble polymers, of caprolactams, pullulan, pectin, mannan and galactomannans, glucomannans and their compounds.

Preferably, the water-soluble polymer may be a cellulose polymer, in particular hydroxypropyl cellulose or hydroxypropyl methyl cellulose, or else an alginate, especially sodium alginate.

The composition according to the invention may be in the form of an emulsion and may also contain specific emulsifiers capable of stabilizing the C-glycoside compounds and may advantageously confer outstanding sensory properties, such as a light and fresh feel.

As examples of particular emulsifiers, mention may especially be made of:
emulsifiers based on polyolefins having a polar part, in a water-in-oil emulsion; and
amphiphilic polymers comprising 2-acryl-amido-2-methylpropanesulphonic acid (AMPS) units, in an oil-in-water emulsion.

### g) Emulsifiers Based on Polyolefins Having a Polar Part

The invention therefore also relates to a composition in the form of a water-in-oil emulsion comprising at least one C-glycoside compound (I) or (II) and at least one polyolefin having a polar part.

The polyolefins having a polar part contribute towards stabilizing said C-glycoside compound in the physiologically acceptable medium of the composition.

The polyolefins having polar part(s) used in the composition of the invention are generally composed of an apolar polyolefin part and at least one polar part. The apolar polyolefin part comprises at least 40 carbon atoms and preferably from 60 to 700 carbon atoms. This apolar part may be chosen from polyolefins such as oligomers, polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. These polyolefins may or may not be hydrogenated.

The polar part of the polyolefins having a polar part may be anionic, cationic, non-ionic, zwitterionic or amphoteric. It is, for example, formed from polyalkylene glycols (especially polyoxyethylene glycols) or from polyalkylene imines, or else from carboxylic acids or diacids, their anhydrides or their compounds such as their esters, their amides and their salts, and blends thereof. Polyolefins having a carboxylic acid polar part may be, for example, derived from the reaction between a polyolefin and at least one carboxylic acid or anhydride that is optionally completely or partially salified, chosen from the group comprising succinic acid or anhydride, maleic acid, maleic anhydride, fumaric acid, itaconic acid, citraconic acid (or methylmaleic acid), mesaconic acid (or methylfumaric acid), aconitic acid, their ester or amide compounds, and mixtures thereof.

Preferably, the polar part of the polyolefin is chosen from the group comprising polyoxyethylene, succinic acid or anhydride, the esters or amides of succinic acid or anhydride, the alkali-metal or alkaline-earth-metal salts or organic salts of succinic acid or anhydride, or the partial salts of monoesters or monoamides of succinic acid or anhydride.

The polyolefins having a polar part that are preferred in the compositions of the invention are polyolefins having optionally modified succinic ends, as described in Patent EP 1 172 089, incorporated here by reference.

As polyolefins having succinic ends, mention may especially be made of polyisobutylenes having optionally modified esterified succinic ends, especially esterified by diethanolamine, and their salts, especially diethanolamine salts, such as the products sold under the names LUBRIZOL® 2724, LUBRIZOL® 2722 and LUBRIZOL® 5603 by the company Lubrizol.

Another polyolefin having a polar part that is particularly preferred is an ester of triethanolamine-diethanolaminoethyl polyisobutenyl succinate. This product is sold, for example, under the name CHEMCCINATE® 2000 by Chemron.

As the polyolefin having a polar part, it is also possible to use an ester of glyceryl polyisobutenyl succinate, especially that sold under the name CHEMCCINATE® 1000 AF by Chemron.

### h) Amphiphilic Polymers Comprising AMPS Units

According to another embodiment of the invention, the composition according to the invention is in the form of an oil-in-water emulsion and comprises at least one C-glycoside compound and at least one amphiphilic polymer comprising 2-acrylamido-2-methylpropane-sulphonic acid (AMPS) units.

This amphiphilic polymer comprising 2-acrylamido-2-methylpropanesulphonic acid (AMPS) units contributes towards stabilizing said C-glycoside compound in the physiologically acceptable medium of the composition.

As the amphiphilic polymer comprising 2-acrylamido-2-methylpropanesulphonic acid (AMPS) units that can be used in the compositions of the invention, mention may be made of those described in Patent Application EP 1 466 587, incorporated here by reference.

As preferred amphiphilic polymers, mention may be made of the copolymers of AMPS and oxyethylenated C₁₂-C₁₄ alcohol methacrylate, especially comprising from 7 to 23 oxyethylenated groups.

To improve the sensory properties of the composition, such as a fresh effect and a feel that is neither sticky nor greasy, in particular for oil-in-water emulsions, tetrapolymers may also be used.

The tetrapolymer used according to the invention comprises, as monomers, methacrylic acid, methylmethacrylate, butylacrylate and a C₁₅-C₂₀ alkyl (meth)acrylate.

Tetrapolymers as defined previously, and also their preparation method, are in particular described in Application US 2003/0021847.

These tetrapolymers may be prepared by emulsion polymerization of the monomers indicated above in the presence of a free-radical initiator such as hydrogen peroxide, tert-butyl hydroperoxide, or sodium, potassium, lithium or ammonium persulphate, the initiator optionally being combined with a reducing agent in order to form a redox system and with a catalyst composed of a transition metal such as a copper or iron salt. The reaction may, for example, be carried out at a temperature between 10 and 120° C., preferably around 85° C., for an approximate duration of three hours.

A tetrapolymer of this type is in particular available from Rohm & Haas under the trade name ALLIANZ OPT in the form of a hydroglycolic dispersion having 48% of active material.

The amount of tetrapolymer (as active material) in the composition according to the invention may range, for example, from 0.1 to 5 wt %, preferably from 0.2 to 5 wt %, better from 0.2 to 2 wt %, and better still from 0.5 to 1 wt % of tetrapolymer, relative to the total weight of the composition.

### Galenic

As specified previously, the compositions according to the invention comprise a physiologically acceptable medium, that is to say a medium that is non-toxic and capable of being applied to the keratinous substances of humans and that preferably has a pleasant appearance, odour and feel.

The compositions according to the invention as defined previously and according to the chosen embodiment (choice of ingredient favouring the solubilization and/or stabilization of said C-glycoside compounds), may be in any galenic form including those conventionally used for a topical application and especially in the form of aqueous or aqueous-alcoholic solutions, oil-in-water (O/W) or water-in-oil (W/O) or multiple (triple: W/O/W or O/W/O) emulsions, aqueous gels, or dispersions of a fatty phase in an aqueous phase using polymeric nanoparticles such as nanospheres and nanocapsules, or ionic and/or non-ionic type lipid vesicles (liposomes, niosomes, oleosomes), nanoemulsions, or thin films. It is also possible to use a composition according to the invention in the form of a dual water and oil phase.

These compositions according to the invention can be prepared according to normal methods available in the art, such preparation being within the skill of the ordinary artisan in view of this disclosure.

In addition, the compositions used according to the invention may be more or less liquid and have the appearance of a white or coloured cream, an ointment, a milk, a lotion, a serum, a paste or a mousse. They may optionally be applied to the skin in the form of an aerosol. They may also be in solid form, and for example in stick form.

When the composition used according to the invention comprises an oily phase, this preferably contains at least one oil. It may contain, in addition, other fatty substances.

As the oils that can be used in the composition of the invention, mention may, for example, be made of:
hydrocarbon-based oils of animal origin, such as perhydrosqualene;
hydrocarbon-based oils of plant origin, such as liquid triglycerides of fatty acids comprising from 4 to 10 carbon atoms such as heptanoic or octanoic acid triglycerides or else, for example sunflower, maize, soybean, pumpkin, grape seed, sesame, hazelnut, apricot, macadamia, arara, sunflower, caster oil and avocado oils, caprylic/capric acid triglycerides such as those sold by Stearineries Dubois or those sold under the names MIGLYOL 810, 812 and 818 by Dynamit Nobel, jojoba oil, shea butter oil;
synthetic esters and ethers, especially of fatty acids, such as oils of formulae R¹COOR² and R¹OR² in which R¹ represents the residue of a fatty acid comprising 8 to 29 carbon atoms, and R² represents a branched or unbranched hydrocarbon-based chain containing 3 to 30 carbon atoms, such as for example purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, such as propylene glycol dioctanoate, neopentyl glycol diheptanoate and diethylene glycol diisononanoate; and pentaerythritol esters such as pentaerythrityl tetraisostearate;
linear or branched hydrocarbons, of mineral or synthetic origin, such as volatile or non-volatile paraffin oils and their compounds, petroleum jelly, polydecenes, hydrogenated polyisobutene such as parleam oil;
fatty alcohols having from 8 to 26 carbon atoms, such as cetyl alcohol, stearyl alcohol and their blend (cetostearyl alcohol), octyldodecanol, 2-butyloctanol, 2-hexyldecanol, 2-undecylpentadecanol, oleyl alcohol or linoleyl alcohol;
partially hydrocarbon-based and/or silicone-based fluoro oils such as those described in document JP-A-2 295 912;
silicone oils such as volatile or non-volatile polydimethylsiloxanes (PDMS) having linear or cyclic silicone-based chains that are liquid or pasty at room temperature, especially cyclopolydimethylsiloxanes (cyclomethicones) such as cyclohexasiloxane; poly-dimethylsiloxanes comprising alkyl, alkoxy or phenyl groups, which are pendent or at the end of the silicone-based chain, groups having 2 to 24 carbon atoms; phenylsilicones such as phenyltrimethicones, phenyldimethicones, phenyltrimethylsiloxydiphenyl-siloxanes, diphenyldimethicones, diphenylmethyl-diphenyltrisiloxanes, 2-phenylethyltrimethylsiloxy-silicates, and polymethylphenylsiloxanes; and mixtures thereof.

The term “hydrocarbon-based oil” in the aforementioned list of oils is understood to mean any oil composed mainly of carbon and hydrogen atoms, and optionally of ester, ether, fluoro, carboxylic acid and/or alcohol groups.

The other fatty substances which may be present in the oily phase are, for example, fatty acids comprising from 8 to 30 carbon atoms, such as stearic acid, lauric acid, palmitic acid and oleic acid; waxes such as lanolin, beeswax, carnauba or candelilla wax, paraffin and montan waxes or microcrystalline waxes, ceresin or ozokerite, synthetic waxes such as polyethylene waxes, Fischer-Tropsch waxes; silicone resins such as trifluoromethyl-C₁₋₄-alkyldimethicone and trifluoro-propyldimethicone; and silicone elastomers such as the products sold under the name KSG by Shin-Etsu, under the names TREFIL, BY29 or EPSX by Dow Corning or under the name GRANSIL by Grant Industries.

These fatty substances may be chosen in a varied manner by a person skilled in the art in order to prepare a composition having the desired properties, for example consistency or texture properties.

According to a particular embodiment of the invention, the composition according to the invention is a water-in-oil (W/O) or oil-in-water (O/W) emulsion. The proportion of the oily phase of the emulsion may range from 5 to 80 wt %, and preferably from 5 to 50 wt %, relative to the total weight of the composition.

The emulsions generally contain at least one emulsifier chosen from amphoteric, anionic, cationic or non-ionic emulsifiers, used alone or as a mixture, and optionally a co-emulsifier. The emulsifiers are chosen in a suitable manner according to the emulsion to be obtained (W/O or O/W). The emulsifier and the co-emulsifier are generally present in the composition, in an amount ranging from 0.3 to 30 wt %, and preferably from 0.5 to 20 wt % relative to the total weight of the composition.

For W/O emulsions, as emulsifiers, mention may be made, for example, of dimethicone copolyols such as the blend of cyclomethicone and dimethicone copolyol, sold under the name DC 5225 C by Dow Corning, and alkyldimethicone copolyols such as laurylmethicone copolyol sold under the name “Dow Corning 5200 Formulation Aid” by Dow Corning and cetyldimethicone copolyol sold under the name ABIL EM 90® by Goldschmidt.

It is also possible to use, as a surfactant for W/O emulsions, a crosslinked solid organopolysiloxane elastomer comprising at least one oxyalkylene group, such as those obtained according to the procedure in Examples 3, 4 and 8 of document U.S. Pat. No. 5,412,004 and the examples from document U.S. Pat. No. 5,811,487, especially the product from Example 3 (synthetic example) from U.S. Pat. No. 5,412,004 and that sold under the reference KSG 21 by Shin Etsu. Other types of KSGs sold by Shin Etsu may also be used, such as KSG-16.

For O/W emulsions, as emulsifiers, mention may be made, for example, of non-ionic emulsifiers such as oxyalkylenated (more particularly polyoxyethylenated) fatty acid and glycerol esters; oxyalkylenated fatty acid and sorbitan esters; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof such as the mixture of glyceryl stearate and PEG-40 stearate.

In a known manner, the cosmetic or dermatological composition of the invention may also contain customary adjuvants used in the cosmetic or dermatological field, such as hydrophilic or lipophilic gelling agents, preservatives, solvents, fragrances, fillers, UV screening agents, bactericides, odour absorbers, colouring materials, plant extracts, salts, antioxidants, basic agents, acids, non-ionic, anionic or cationic surfactants, mother of pearl and particles.

The amounts of these various adjuvants are those conventionally used in the field considered, and are for example from 0.01 to 20% of the total weight of the composition. These adjuvants, depending on their nature, may be introduced into the fatty phase, into the aqueous phase and/or into the lipid vesicles.

As fillers that can be used in the composition of the invention, mention may be made, for example, besides pigments, of silica powder, a colloidal amorphous silica; talc; polyamide particles and especially those sold under the name ORGASOL by Atochem; polyethylene powders; microspheres based on acrylic copolymers, such as those made of the ethylene glycol dimethacrylate/lauryl methacrylate copolymer sold by Dow Corning under the name POLYTRAP; expanded powders such as hollow microspheres and especially the microspheres sold under the name EXPANCEL by Kemanord Plast or under the name MICROPEARL F 80 ED by Matsumoto; silicone resin microbeads such as those sold under the name TOSPEARL by Toshiba Silicone; and mixtures thereof. These fillers may be present in amounts ranging from 0 to 20 wt % and preferably from 1 to 10 wt % relative to the total weight of the composition.

As hydrophilic or lipophilic gelling agents, mention may especially be made of CARBOPOL, LUVIGEL, HOSTACERIN AMPS, SIMULGEL, SEPIGEL type acrylamide gelling agents such as SEPIGEL 305® from Seppic, xanthan, guar and cellulose gums, alginates and mixtures thereof. Hectorites may also be mentioned.

As surfactants, foaming surfactants will preferably be used.

Foaming surfactants are detergents and differ from emulsifying surfactants in the value of their HLB (hydrophilic lipophilic balance), the HLB being the ratio between the hydrophilic part and the lipophilic part in the molecule. The term “HLB” is well known to a person skilled in the art and is described, for example, in “The HLB system. A time-saving guide to Emulsifier Selection” (published by ICI Americas Inc.; 1984).

For emulsifying surfactants, the HLB generally ranges from 3 to 8 for the preparation of water-in-oil (W/O) emulsions and from 8 to 18 for the preparation of oil-in-water (O/W) emulsions, whereas foaming surfactants generally have an HLB above 20.

Advantageously, the compositions according to the invention may comprise a surfactant having an HLB above 20.

The surfactant may be present in a composition according to the invention in an amount ranging from 0.1 to 50 wt %, preferably from 0.5 to 20 wt %, in particular from 1 to 15 wt %, or even from 5 to 10 wt % relative to the total weight of the composition.

a) Non-ionic surfactants may be chosen, for example, from alkyl polyglucosides (APGs), maltose esters, polyglycerolated fatty alcohols, glucamine compounds such as 2-ethylhexyloxycarbonyl-N-methylglucamine, and mixtures thereof.

As alkyl polyglucosides, use is preferably made of those containing an alkyl group comprising from 6 to 30 carbon atoms and preferably from 8 to 16 carbon atoms and containing a hydrophilic (glucoside) group preferably comprising 1, 2 or 3 glucoside units. As alkyl polyglucosides, mention may be made, for example, of decyl glucoside (Alkyl C9/C11 polyglucoside (1.4)) such as the product sold under the name MYDOL 10® by Kao Chemicals, the product sold under the name PLANTAREN 2000 UP® by Cognis and the product sold under the name ORAMIX NS 10® by Seppic; caprylyl/capryl glucoside such as the product sold under the name ORAMIX CG 110® by Seppic; lauryl glucoside such as the products sold under the names PLANTAREN 1200 N® and PLANTACARE 1200® by Cognis; coco glucoside such as the product sold under the name PLANTACARE 818/UP® by Cognis; and mixtures thereof.

The maltose compounds are, for example, those described in document EP-A-566 438, such as O-octanoyl-6′-D-maltose, or else O-dodecanoyl-6′-D-maltose described in document FR-2,739,556.

Among the polyglycerolated fatty alcohols, mention may be made of polyglycerolated (3.5 mol of glycerol) dodecanediol, a product sold under the name CHIMEXANE NF® by Chimex.

b) Anionic surfactants may be chosen, for example, from soaps (alkali metal salts of fatty acids), carboxylates, acylamino acids, amidoether carboxylates, alkyl polyaminocarboxylates, alkyl ether sulphates such as sodium laureth sulphates, alkyl sulphonates, isethionates, alkyl methyl taurates, alkyl sulphosuccinates, alkyl sulphoacetates, alkyl phosphates (monoalkyl or dialkyl phosphates), salts thereof and mixtures thereof.

As carboxylates, mention may especially be made of alkyl glycol carboxylic acids (or 2-(2-hydroxyalkyloxy)acetic acids), and their salts such as, for example, sodium lauryl glycol carboxylate, sold under the names BEAULIGHT SHAA® and BEAULIGHT LCA-25N® by Sanyo (CTFA name: Sodium Lauryl Glycol Carboxylate), or its corresponding acid form sold under the name BEAULIGHT SHAA (Acid Form)® by Sanyo.

As acylamino acids, mention may be made, for example, of sodium cocoyl glycinate sold by Ajinomoto under the name AMILITE GCS 12, sodium lauroyl glutamate sold by Ajinomoto under the name AMISOFT LS11 and sodium lauroyl sarcosinate sold by Seppic under the name ORAMIX L 30.

As alkyl phosphates, mention may be made, for example, of lauryl phosphate sold by Kao under the name MAP 20.

c) Amphoteric and zwitterionic foaming surfactants may be chosen, for example, from betaine compounds, including amidopropyl betaines, amphoacetates and amphodiacetates, hydroxysultaines and mixtures thereof.

As betaine compounds, mention may be made, for example, of coco betaine, such as the product sold under the name DEHYTON AB-30® by Cognis; lauryl betaine such as the product sold under the name GENAGEN KB® by Clariant; oxyethylenated (10 EO) lauryl betaine, such as the product sold under the name LAURYL ETHER (10 EO) BETAINE® by Shin Nihon Rica; oxyethylenated (10 EO) stearyl betaine such as the product sold under the name STEARYL ETHER (10 EO) BETAINE® by Shin Nihon Rica; cocamidopropyl betaine sold, for example, under the name VELVETEX BK 35® by Cognis; undecyleneamidopropyl betaine sold, for example, under the name AMPHORAM U® by Ceca; and mixtures thereof.

As amphoacetates and amphodiacetates, mention may be made, for example, of N-disodium N-cocoyl-N-carboxy-methoxyethyl-N-carboxymethylethylenediamine (CTFA name: disodium cocamphodiacetate) such as the product sold under the name MIRANOL C2M CONCENTRE NP® by Rhodia Chimie; N-sodium N-cocoyl-N-hydroxyethyl-N-carboxy-methylethylenediamine (CTFA name: sodium cocamphoacetate) and mixtures thereof.

According to one embodiment of the invention, a surfactant suitable for the invention may be chosen from alkyl polyglucosides, betaine compounds, alkyl glycol carboxylic acids and their salts, alkyl ether sulphates, alkyl phosphates, amphodiacetates, ampho-acetates, alkyl glycinates, acyl glutamates, acyl sarcosinates and mixtures of the latter.

According to one embodiment, a surfactant suitable for the invention may be chosen from decyl glucoside, cocoyl glucoside, sodium lauryl ether sulphate, cocoyl betaine, lauroyl betaine, cocamidopropyl betaine, lauramido propyl betaine, lauryl glycol carboxylate, cocoampho(di)acetate, lauroampho(di)acetate, potassium lauryl phosphate, and a mixture of the latter.

According to one particular embodiment, when the composition contains a foaming surfactant, it does not contain an oxyalkylenated compound.

The use of a C-glycoside compound according to the invention in combination with at least one foaming surfactant improves the quality of the foam of said compositions containing at least one foaming surfactant.

This combination is especially advantageous in cleansing and/or makeup removing compositions.

As preferred foaming surfactants, mention may be made of:
sodium cocoyl isethionate, such as the sodium cocoyl isethionate/sodium isethionate mixture sold by BASF under the name JORDAPON CI P®;
decyl glucoside, such as PLANTACARE 2000 UP® sold by Cognis;
potassium laurate, such as LAURATE DE POTASSIUM DUB LK® sold by Stearinerie Dubois;
sodium lauroyl oat amino acids, such as the 30% aqueous solution of lauroyl oat amino acids, in the form of the protected (1.4% of phenonip) sodium salt sold under the name PROTEOL OAT® by Seppic;
potassium lauroyl wheat amino acids, such as the stabilized (phenonip-potassium sorbate 0.4-0.4%) potassium lauroyl wheat protein hydrolysed in water at a concentration of 25%, sold under the name AMINOFOAM W OR® by Croda;
polyglyceryl-3 hydroxylauryl ether or polyglycerolated (3.5 mol of glycerol) dodecanediol, such as CHIMEXANE® by Chimex;
sodium lauryl ether sulphate, such as TEXAPON AOS 225 UP® by Cognis;
N-disodium N-cocoyl-N-carboxymethoxyethyl-N-carboxy-methylethylenediamine (CTFA name: disodium cocamphodiacetate) such as the product sold under the name MIRANOL C2M CONCENTRE NP® by Rhodia Chimie;
coco betaine or cocoyl betaine in aqueous solution, such as MIRATAINE BB/FLA by Rhodia;
PEG-7 glyceryl cocoate or oxyethylenated (7 EO) glyceryl cocoate, such as CETIOL HE® by Cognis;
lauryl betaine such as the 30% aqueous solution of lauryl dimethylamine betaine sold under the name EMPIGEN BB/LS® by Huntsman;
sodium cocamphoacetate, such as the 32% aqueous solution of sodium N-cocylamidoethyl-N-hydroxyethyl glycinate sold under the name MIRANOL ULTRA C 32® by Rhodia;
triethanolamine lauryl sulphate or TEA lauryl sulphate, such as the 40% aqueous solution of triethanolamine (C12/C14 70/30) lauryl sulphate sold under the name TEXAPON T 42® by Cognis;
sodium lauryl glycol carboxylate, sold under the names BEAULIGHT SHAA and BEAULIGHT LCA-25N® by Sanyo (CTFA name: Sodium Lauryl glycol Carboxylate) or its corresponding acid form sold under the name BEAULIGHT SHAA (Acid Form)® by Sanyo;
lauryl phosphate sold by Kao under the name MAP 20®; and
sodium cocoyl glycinate, such as AMILITE GCS® 12 by Ajinomoto.

According to one particular embodiment, the composition according to the invention comprises at least one C-glycoside compound and at least sodium cocoyl isethionate, such as the sodium cocoyl isethionate/sodium isethionate mixture sold by BASF under the name JORDAPON CI P®.

According to another embodiment, the composition according to the invention comprises at least one C-glycoside compound and at least decyl glucoside, such as PLANTACARE 2000 UP® sold by Cognis, said composition not comprising an oxyalkylenated compound.

According to another embodiment, the composition according to the invention comprises at least one C-glycoside compound and at least potassium laurate, such as LAURATE DE POTASSIUM DUB LK® sold by Stearinerie Dubois.

According to another embodiment, the composition according to the invention comprises at least one C-glycoside compound and at least sodium lauryl oat amino acids, such as the 30% aqueous solution of lauroyl oat amino acids, in the form of the protected (1.4% of phenonip) sodium salt sold under the name PROTEOL OAT® by Seppic.

According to one particular embodiment, the composition according to the invention comprises at least one C-glycoside compound and at least potassium lauroyl wheat amino acids, such as the stabilized (phenonip-potassium sorbate 0.4-0.4%) potassium lauroyl wheat protein hydrolysed in water at a concentration of 25% sold under the name AMINOFOAM W OR® by Croda.

According to another embodiment, the composition according to the invention comprises at least one C-glycoside compound and at least polyglyceryl-3-hydroxylauryl ether or polyglycerolated (3.5 mol of glycerol) dodecanediol such as CHIMEXANE® by Chimex, said composition not comprising an oxyalkylenated compound.

According to one particular embodiment, the composition according to the invention comprises at least one C-glycoside compound and at least one sodium lauryl ether sulphate, such as TEXAPON AOS 225 UP® by Cognis, said composition not comprising an oxyalkylenated compound.

According to another embodiment, the composition according to the invention comprises at least one C-glycoside compound and at least one N-disodium N-cocoyl-N-carboxymethoxyethyl-N-carboxymethylethylene-diamine (CTFA name: disodium cocamphodiacetate) such as the product sold under the name MIRANOL C2M CONCENTRE NP® by Rhodia Chimie, said composition not comprising an oxyalkylenated compound.

According to another embodiment, the composition according to the invention comprises at least one C-glycoside compound and at least one coco betaine or cocoyl betaine in aqueous solution, such as MIRATAINE BB/FLA by Rhodia, said composition not comprising an oxyalkylenated compound.

According to another embodiment, the composition according to the invention comprises at least one C-glycoside compound and at least PEG-7 glyceryl cocoate or oxyethylenated (7 EO) glyceryl cocoate such as CETIOL HE® by Cognis.

According to one particular embodiment, the composition according to the invention comprises at least one C-glycoside compound and at least one lauryl betaine such as the 30% aqueous solution of lauryl dimethylamine betaine sold under the name EMPIGEN BB/LS® by Huntsman, said composition not comprising an oxyalkylenated compound.

According to another embodiment, the composition according to the invention comprises at least one C-glycoside compound and at least sodium cocoamphoacetate, such as the 32% aqueous solution of sodium N-cocylamidoethyl-N-hydroxyethyl glycinate sold under the name MIRANOL ULTRA C 32® by Rhodia, said composition not comprising an oxyalkylenated compound.

According to another embodiment, the composition according to the invention comprises at least one C-glycoside compound and at least triethanolamine lauryl sulphate or TEA lauryl sulphate, such as the 40% aqueous solution of triethanolamine (C12/C14 70/30) lauryl sulphate sold under the name TEXAPON T 42® by Cognis.

According to another embodiment, the composition according to the invention comprises at least one C-glycoside compound and at least sodium lauryl glycol carboxylate sold under the names BEAULIGHT SHAA® and BEAULIGHT LCA-25N® by Sanyo (CTFA name: Sodium Lauryl Glycol Carboxylate) or its corresponding acid form sold under the name BEAULIGHT SHAA (Acid Form)® by Sanyo, said composition not comprising an oxyalkylenated compound.

According to one particular embodiment, the composition according to the invention comprises at least one C-glycoside compound and at least lauryl phosphate sold by Kao under the name MAP 20®, said composition not comprising an oxyalkylenated compound.

According to another embodiment, the composition according to the invention comprises at least one C-glycoside compound and at least sodium cocoyl glycinate, such as AMILITE GCS® 12 by Ajinomoto, said composition not comprising an oxyalkylenated compound.

The use of a C-glycoside compound according to the invention in combination with at least one filler, improves the dispersing effect of the fillers in said compositions containing at least one filler.

As preferred fillers, mention may especially be made of:
hollow hemispherical silicone resin particles such as the NLK 500®, NLK 506® and NLK 510® particles from Takemoto Oil and Fat, especially described in EP-A-1 579 849;
polyamide fibres, such as Nylon® fibres, in particular nylon-6,6 fibres sold under the name PULPE POLYAMIDE12185 by Utexbel or FIBERLON 931-D1-S by LCW;
microencapsulated iron oxides, in particular:
   microencapsulated yellow iron oxides, such as the mixture of iron oxides, titanium oxides, boron nitride, acrylates/ammonium methacrylate copolymer, and triethyl citrate, sold under the name YELLOWCAP1® by Tagra Biotechnologies LTD;
   microencapsulated red iron oxides, such as the mixture of iron oxides, titanium oxides, boron nitride, acrylates/ammonium methacrylate copolymer, and triethyl citrate, sold under the name REDCAP1® by Tagra Biotechnologies LTD;
   microencapsulated black iron oxides, such as the mixture of iron oxides, titanium oxides, boron nitride, acrylates/ammonium methacrylate copolymer, and triethyl citrate, sold under the name BLACKCAP1® by Tagra Biotechnologies LTD.

According to one particular embodiment, the composition according to the invention contains at least one C-glycoside compound and at least hollow hemispherical silicone resin particles such as the NLK 500®, NLK 506® and NLK 510® particles by Takemoto Oil and Fat, especially described in EP-A-1 579 849.

According to another embodiment, the composition according to the invention contains at least one C-glycoside compound and at least polyamide fibres, such as Nylon® fibres, in particular nylon-6,6 fibres sold under the name PULPE POLYAMIDE12185 by Utexbel or FIBERLON 931-D1-S by LCW.

According to another embodiment, the composition according to the invention contains at least one C-glycoside compound and at least microencapsulated iron oxides, in particular:
microencapsulated yellow iron oxides, such as the mixture of iron oxides, titanium oxides, boron nitride, acrylates/ammonium methacrylate copolymer, and triethyl citrate, sold under the name YELLOWCAP1® by Tagra Biotechnologies LTD;
microencapsulated red iron oxides, such as the mixture of iron oxides, titanium oxides, boron nitride, acrylates/ammonium methacrylate copolymer, and triethyl citrate, sold under the name REDCAP1® by Tagra Biotechnologies LTD; or
microencapsulated black iron oxides, such as the mixture of iron oxides, titanium oxides, boron nitride, acrylates/ammonium methacrylate copolymer, and triethyl citrate, sold under the name BLACKCAP1® by Tagra Biotechnologies LTD.

The combination between a C-glycoside compound and fillers will be especially advantageous in skincare and/or makeup compositions.

As antioxidants, mention may especially be made of polyphenols, tannic acid, epigallocatechins and the natural extracts containing them, anthocyanins, rosemary extracts, olive leaf extracts, green tea, resveratrol and its compounds, pycnogenol, ergothineine, N-acetylcysteine, biotin, chelating agents, idebenone, plant extracts such as PRONALEN BIOPROTECT™ from Provital, anti-free radical agents such as vitamin E, co-enzyme Q10, bioflavonoids, SOD, phytantriol, lignans, melatonin, pidolates, gluthatione.

According to a preferred embodiment of the invention, the composition used according to the invention contains at least one UV screening agent (or sunscreen) which may be a chemical screening agent or a physical screening agent or a mixture of such screening agents.

By way of illustration and in a non-limiting way, the following families may be mentioned (the names corresponding to the CTFA nomenclature for screening agents):

anthranilates, in particular menthyl anthranilate; benzophenones, in particular benzophenone-1, benzophenone-3, benzophenone-5, benzophenone-6, benzophenone-8, benzophenone-9, benzophenone-12, and preferentially benzophenone-2 (oxybenzone) or benzophenone-4 (UVINUL MS40 available from BASF); benzylidenecamphors, in particular 3-benzylidene-camphor, benzylidenecamphorsulphonic acid, camphor benzalkonium methosulphate, polyacrylamidomethyl-benzylidene camphor, terephthalylidenedicamphor-sulphonic acid, and preferentially 4-methylbenzylidene camphor (EUSOLEX 6300 available from Merck); benzimidazols, in particular benzimidazilate (NEO HELIOPAN AP available from Haarmann and Reimer), or phenylbenzimidazolsulphonic acid (EUSOLEX 232 available from Merck); benzotriazols, in particular drometrizole trisiloxane or methylenebis[(benzotriazolyl)(tetra-methylbutyl)phenol] (TINOSORB M available from Ciba); cinnamates, in particular cinoxate, DEA methoxycinnamate, diisopropyl methylcinnamate, glyceryl-ethylhexanoate dimethoxycinnamate, isopropyl methoxy-cinnamate, isoamyl cinnamate and preferentially ethocrylene (UVINUL N35 available from BASF), octyl methoxycinnamate (PARSOL MCX available from Hoffmann La Roche), or octocrylene (UVINUL 539 available from BASF); dibenzoylmethanes, in particular butyl methoxy-dibenzoylmethane (PARSOL 1789); imidazolines, in particular ethylhexyl dimethoxybenzylidene dioxo-imidazoline; PABAs, in particular ethyldihydroxypropyl PABA, ethylhexyldimethyl PABA, glyceryl PABA, PABA, PEG-25 PABA, and preferentially diethylhexyl butamido triazone (UVASORB HEB available from 3V Sigma), ethylhexyl triazone (UVINUL T150 available from BASF), or ethyl PABA (benzocaine); salicylates, in particular dipropylene glycol salicylate, ethylhexyl salicylate, homosalate, or TEA salicylate; triazines, in particular anisotriazine (TINOSORB S available from Ciba); drometrizole trisiloxane, zinc oxide, titanium dioxide, and coated or uncoated zinc, iron, zirconium or cerium oxides.

The amount of screening agents depend on the final desired use. It may range, for example, from 1 to 20 wt % and better from 2 to 10 wt % relative to the total weight of the composition.

The compositions according to the invention could be applied directly onto the skin or, alternatively, onto cosmetic or dermatological supports of the occlusive or non-occlusive type, intended to be applied to the skin in a localized fashion.

The support may be an “occlusive” support. By way of example, the support is formed from a thermoplastic, chosen from high- and low-density polyethylenes, polypropylenes, polyvinyl chlorides, ethylene/vinyl acetate copolymers, polyesters and polyurethanes, or from a complex of such materials. These materials may also be present in laminated form with at least one metal sheet, such as an aluminium foil.

The support layer may be of any appropriate thickness which will provide the desired support and protective functions. Preferably, the thickness of the support layer is between about 20 μm and about 1.5 mm. Advantageously, the support layer is sufficiently flexible so as to be able to perfectly fit the shape of the skin, and so as not to cause a feeling of discomfort in the user.

Preferably however, the support is “non-occlusive”. In the latter assumption, a support is advantageously used that is formed from a paper, a porous or perforated thermoplastic, a woven fabric, a non-woven fabric or a perforated non-woven fabric.

The C-glycoside compound according to the invention could be used according to the invention as an antioxidant.

The C-glycoside compound as it is and/or the composition containing it could be used according to the invention to improve the appearance and/or visibility of the pores, in particular by reducing the size and/or number of pores visible on the skin of the face, and especially in the T zone (forehead, nose, cheeks, chin), in particular on the nose and cheeks.

According to one particular embodiment, the composition according to the invention will moreover comprise at least one other active agent chosen from a black tea extract, a brown sugar extract, a lychee extract and mixtures thereof. In particular, said combination and/or the composition containing it is used according to the invention to improve the tightening of the pores of the skin, in particular on the face and especially in the T zone (forehead, nose, cheeks, chin), in particular on the nose and cheeks.

The C-glycoside compound as it is and/or the composition containing it could be used according to the invention to prevent and/or treat ageing of the area around the lips, in particular the top lip area.

According to one particular embodiment, the C-glycoside compound could be used in combination with at least one agent chosen from cinnamic acid, phloroglucinol, a soybean extract and mixtures thereof. Preferably, the C-glycoside compound is combined with a phytocomplex comprising cinnamic acid, phloroglucinol and a soybean extract.

This combination will be especially advantageous for preventing and/or treating hormonal ageing of the area around the lips, in particular of the top lip area.

According to another embodiment of the invention, said compositions according to the invention may be combined with compositions administered orally, containing additional cosmetic active agents having a beneficial effect on the appearance of the skin, such as for example additional active agents intended to combat the signs of skin ageing or additional active agents intended to combat greasy skin.

The additional ingredient used in the composition of the invention may also be a cosmetic or pharmaceutical ingredient and/or active agent, in particular ingredients and/or active agents that are beneficial for the appearance and/or texture of the skin.

According to one particular embodiment of the invention, the composition according to the invention comprises at least one ingredient for solubilizing and/or stabilizing said C-glycoside compound and, in addition, at least one other additional ingredient and/or active agent chosen from a cosmetic or pharmaceutical ingredient and/or active agent.

### C—Additional Cosmetic and Dermatological Active Agents

The additional active agents may especially be chosen from moisturizers, desquamating agents, agents improving the barrier function, depigmenting agents, antioxidants, dermo-decontracting agents, antiglycation agents, agents stimulating the synthesis of dermal and/or epidermal macromolecules and/or preventing their degradation, agents stimulating the proliferation of fibroblasts or keratinocytes and/or the differentiation of the keratinocytes, agents favouring maturing of the horny envelope, NO-synthase inhibitors, peripheral benzodiazepine receptor (PBR) antagonists, agents increasing the activity of the sebaceous gland, agents stimulating cell energy metabolism, tightening agents, lipo-restructuring agents, slimming agents, agents favouring skin microcirculation, soothing and/or anti-irritant agents, sebo-regulating or antiseborrhoeic agents, astringents, wound-healing agents, anti-inflammatory agents and anti-acne agents.

A person skilled in the art will choose said active agent or agents as a function of the desired effect on the keratinous substances.

For caring for and/or making up aged skin, he will preferably choose at least one active agent chosen from moisturizers, desquamating agents, agents improving the barrier function, depigmenting agents, antioxidants, dermo-decontracting agents, antiglycation agents, agents stimulating the synthesis of dermal and/or epidermal macromolecules and/or preventing their degradation, agents stimulating the proliferation of fibroblasts or keratinocytes and/or the differentiation of the keratinocytes, agents favouring maturing of the horny envelope, NO-synthase inhibitors, peripheral benzodiazepine receptor (PBR) antagonists, agents increasing the activity of the sebaceous gland, agents stimulating cell energy metabolism, lipo-restructuring agents, and agents favouring skin microcirculation for the area around the eyes.

The composition could moreover comprise at least one ingredient such as fillers with a soft-focus effect or agents favouring the natural coloration of the skin, intended to complement the biological effect of these active agents or to provide an immediate visual anti-ageing effect.

For caring for and/or making up greasy skin, a person skilled in the art will preferably choose at least one active agent chosen from desquamating agents, sebo-regulating agents or antiseborrhoeic agents and astringents.

For caring for and/or making up acne-prone skin, he will preferably choose at least one active agent chosen from anti-acne agents, wound-healing agents and anti-inflammatory agents.

For slimming care of the body, he will preferably choose an active agent chosen from slimming agents and agents favouring skin microcirculation.

The composition could moreover comprise at least one additional ingredient intended to complement the biological effect of these active agents or to provide an immediate visual effect; mention may especially be made of matifying agents, fillers with a soft-focus effect, fluorescent agents, agents favouring the naturally pink coloration of the skin and abrasive or exfoliant fillers.

According to a first embodiment, the composition according to the invention comprises at least one moisturizer.

According to another embodiment, the composition according to the invention comprises at least one desquamating agent.

According to another embodiment, the composition according to the invention comprises at least one agent that improves the barrier function.

In particular, said composition between the C-glycoside compound and the agent improving the barrier function and/or said composition containing it is used according to the invention for improving the barrier function and/or the wound-healing process of the skin.

According to another embodiment, the composition according to the invention comprises at least one depigmenting agent.

In particular, said combination between the C-glycoside compound and the depigmenting agent and/or said composition containing it is used according to the invention to favour the whitening and/or depigmentation of the skin.

According to another embodiment, the composition according to the invention comprises at least one antioxidant.

In particular, said combination between the C-glycoside compound and the antioxidant and/or said composition containing it is used according to the invention to improve the antioxidant effect of the composition.

According to another embodiment, the composition according to the invention comprises at least one dermo-decontracting agent.

In particular, said combination between the C-glycoside compound and the dermo-decontracting agent and/or said composition containing it is used according to the invention to favour the dermo-decontraction and/or dermo-relaxation of the skin, and thus to prevent and/or treat the expression lines on the face and in particular on the forehead and in the area between the eyebrows.

According to another embodiment, the composition according to the invention comprises at least one anti-glycation agent.

According to another embodiment, the composition according to the invention comprises at least one agent stimulating the synthesis of dermal and/or epidermal macromolecules and/or preventing their degradation.

According to another embodiment, the composition according to the invention comprises at least one agent stimulating the proliferation of fibroblasts or keratinocytes and/or the differentiation of the keratinocytes.

According to another embodiment, the composition according to the invention comprises at least one agent that favours the maturing of the horny envelope.

According to another embodiment, the composition according to the invention comprises at least one NO-synthase inhibitor.

According to another embodiment, the composition according to the invention comprises at least one peripheral benzodiazepine receptor (PBR) antagonist.

According to another embodiment, the composition according to the invention comprises at least one agent that increases the activity of the sebaceous gland.

According to another embodiment, the composition according to the invention comprises at least one agent that stimulates cell energy metabolism.

According to another embodiment, the composition according to the invention comprises at least one tightening agent.

According to another embodiment, the composition according to the invention comprises at least one lipo-restructuring agent.

In particular, said combination between a C-glycoside compound and a lipo-restructuring agent and/or said composition containing it is used according to the invention to improve the lipostructure of the skin, in particular decreasing the hollowing of the face due to age.

According to another embodiment, the composition according to the invention comprises at least one slimming agent.

According to another embodiment, the composition according to the invention comprises at least one agent that favours skin microcirculation.

In particular, said combination between the C-glycoside compound and the agent favouring skin microcirculation and/or said composition containing it is used according to the invention to improve the appearance of the area around the eye, in particular to reduce dark circles.

According to another embodiment, the composition according to the invention comprises at least one soothing and/or anti-irritant agent.

In particular, said combination between the C-glycoside compound and the soothing and/or anti-irritant agent and/or said composition containing it is used according to the invention to soothe the skin.

According to another embodiment, the composition according to the invention comprises at least one sebo-regulating or antiseborrhoeic agent.

According to another embodiment, the composition according to the invention comprises at least one astringent.

According to another embodiment, the composition according to the invention comprises at least one wound-healing agent.

According to another embodiment, the composition according to the invention comprises at least one anti-inflammatory agent.

According to another embodiment, the composition according to the invention comprises at least one anti-acne agent.

According to another embodiment, the composition according to the invention comprises at least one matifying agent.

According to another embodiment, the composition according to the invention comprises at least one filler with a soft-focus effect.

According to another embodiment, the composition according to the invention comprises at least one fluorescent agent.

According to another embodiment, the composition according to the invention comprises at least one agent favouring the naturally pink coloration of the skin.

According to another embodiment, the composition according to the invention comprises at least abrasive fillers or exfoliating agents.

Examples of such compounds are described below.

### 1. Moisturizing Agents or Humectants

As humectant or moisturizing agents, mention may especially be made of glycerol and its compounds, urea and its compounds, especially HYDROVANCE® sold by National Starch, lactic acids, hyaluronic acid, AHAs, BHAs, sodium pidolate, xylitol, serine, sodium lactate, ectoine and its compounds, chitosan and its compounds, collagen, plankton, an Imperata cylindra extract sold under the name MOIST 24® by Sederma, acrylic acid homopolymers such as LIPIDURE-HM® by NOF Corporation, beta-glucan and in particular sodium carboxymethyl beta-glucan from Mibelle-AG-Biochemistry; a mixture of passion flower, apricot and maize oils, and rice bran sold by Nestlé under the name NutraLipids®; a C-glycoside compound such as those described in Application WO 02/051828 and in particular C-β-D-xylopyranoside-2-hydroxypropane in the form of a 30 wt % solution of active material in a water/propylene glycol mixture (60/40 wt %) such as the product manufactured by Chimex under the trade name MEXORYL SBB®; a musk rose oil sold by Nestlé; a zinc-enriched extract of Prophyridium cruentum microalgae sold by Vincience under the name ALGUALANE ZINC®; spheres of collagen and of chondroitin sulphate of marine origin (atelocollagen) sold by Engelhard Lyon under the name marine filling spheres; hyaluronic acid spheres such as those sold by Engelhard Lyon; and arginine.

Preferably, a moisturizing agent will be used chosen from urea and its compounds especially HYDROVANCE® sold by National Starch, hyaluronic acid, AHAs, BHAs, acrylic acid homopolymers such as LIPIDURE-HM® by NOF Corporation, beta-glucan and in particular sodium carboxymethyl beta-glucan from Mibelle-AG-Biochemistry; a mixture of passion flower, apricot and maize oils, and rice bran sold by Nestlé under the name NutraLipids®; a C-glycoside compound such as those described in Application WO 02/051828 and in particular C-β-D-xylopyranoside-2-hydroxypropane in the form of a 30 wt % solution of active material in a water/propylene glycol mixture (60/40 wt %) such as the product manufactured by Chimex under the trade name MEXORYL SBB®; a musk rose oil sold by Nestlé; a zinc-enriched extract of Prophyridium cruentum microalgae sold by Vincience under the name ALGUALANE ZINC®; spheres of collagen and of chondroitin sulphate of marine origin (atelocollagen) sold by Engelhard Lyon under the name marine filling spheres; hyaluronic acid spheres such as those sold by Engelhard Lyon; and arginine.

### 2. Desquamating Agents

The term “desquamating agent” is understood to mean any compound capable of acting:
either directly on desquamation by favouring exfoliation, such as β-hydroxy acids (BHAs), in particular salicylic acid and its compounds (including 5-(n-octanoyl)salicylic acid otherwise known as capryloyl salicylic acid (INCI name)); α-hydroxy acids (AHAs), such as glycolic acid, citric acid, lactic acid, tartaric acid, malic acid or mandelic acid; 8-hexadecene-1,16-dicarboxylic acid or 9-octadecenedioic acid; urea and its compounds; gentisic acid and its compounds; oligofucoses; cinnamic acid; Saphora japonica extract; resveratrol and some jasmonic acid compounds;
or on the enzymes involved in desquamating or degrading the corneodesmosomes, glycosidases, stratum corneum chymotryptic enzyme (SCCE) or even other proteases (trypsin, chymotrypsin-like). Mention may be made of aminosulphonic compounds and in particular 4-(2-hydroxyethyl)piperazine-1-propanesulphonic acid (HEPES); 2-oxothiazolidine-4-carboxylic acid (procysteine) and its compounds; compounds of glycine-type α-amino acids (as described in EP 0 852 949, and also sodium methylglycinediacetate sold by BASF under the trade name TRILON M); honey; sugar compounds such as O-octanoyl-6-D-maltose and N-acetylglucosamine.

As other desquamating agents that can be used in the composition according to the invention, mention may be made of
oligofructoses, EDTA and its compounds, Laminaria extracts, O-linoleyl-6-D-glucose, (3-hydroxy-2-pentylcyclopentyl)acetic acid, glyceryl trilactate, O-octanyl-6′-D-maltose, S-carboxymethyl cysteine, silicon-containing salicylate compounds such as those described in Patent EP 0 796 861, oligofucases such as those described in Patent EP 0 218 200, 5-acylsalicylic acid salts, active agents having effects on transglutaminase as in Patent EP 0 899 330:
extract of Opuntia ficus indica flower such as Exfolactive® from Silab;
8-hexadecene-1,16-dicarboxylic acid;
esters of glucose and of vitamin F; and
mixtures thereof.

As preferred desquamating agents, mention may be made of β-hydroxy acids, such as 5-(n-octanoyl)salicylic acid; urea; glycolic acid, citric acid, lactic acid, tartaric acid, malic acid or mandelic acid; 4-(2-hydroxyethyl)piperazine-1-propanesulphonic acid (HEPES); Saphora japonica extract; honey; N-acetyl-glucosamine; sodium methylglycinediacetate, and mixtures thereof.

Even more preferentially, a desquamating agent will be used in the compositions of the invention chosen from 5-(n-octanoyl)salicylic acid; urea; 4-(2-hydroxy-ethyl)piperazine-1-propanesulphonic acid (HEPES); Saphora japonica extract; honey; N-acetylglucosamine; sodium methylglycinediacetate, and mixtures thereof.

3. Agents that Improve the Barrier Function

As agents that improve the barrier function, mention may especially be made of arginine, serine, an extract of Thermus thermophilus such as VENUCEANE® from Sederma, an extract of wild yam (Dioscorea villosa) rhizome such as ACTIGEN Y® from Active Organics, plankton extracts such as OMEGA PLANKTON® from Secma, yeast extracts such as RELIPIDIUM® from Coletica, a chestnut extract such as RECOVERINE® from Silab, a cedar bud extract such as GATULINE ZEN® from Gattefossé, sphingosines such as the salicyloyl sphingosine sold under the name PHYTOSPHINGOSINE® SLC by Degussa, a mixture of xylitol, xylityl polyglycoside and xylitan such as AQUAXYL® by Seppic, extracts of solanaceae such as LIPIDESSENCE® from Coletica; unsaturated omega-3 oils such as musk rose oils and mixtures thereof.

Mention may also be especially made of ceramides or compound, in particular ceramides of type 2 (such as N-oleoyldihydrosphinogsine), of type 3 (such as stearoyl-4-hydroxysphinganine (INCI name)) and of type 5 (such as N-2-hydroxypalmitoyldihydrosphingosine, having the INCI name: hydroxypalmitoyl sphinganine), compounds based on sphingoids, glycosphingolipids, phospholipids, cholesterol and its compounds, phytosterols, essential fatty acids, diacyl glycerol, 4-chromanon and chromon compounds, petroleum jelly, lanolin, rhea butter, cocoa butter, lanolin and PCA salts.

As preferred agents having a restructuring effect of the skin barrier, mention will be made of an extract of Thermus thermophilus, an extract of wild yam (Dioscorea villosa) rhizome, a yeast extract, a chestnut extract, a cedar bud extract, arginine, serine, ceramides especially of type 3 and 5, and mixtures thereof.

Preferably, serine, arginine or a mixture thereof will be used.

### 4. Depigmenting Agents

As depigmenting agents, mention may especially be made of vitamin C and its compounds and especially vitamin CG, vitamin CP and 3-O-ethyl-vitamin C, alpha- and beta-arbutin, ferulic acid, lucinol and its compounds, kojic acid, resorcinol and its compounds, tranexamic acid and its compounds, gentisic acid, homogentisate, methyl gentisate or homogentisate, dioic acid, calcium D-pantetheine sulphonate, lipoic acid, ellagic acid, vitamin B3, linoleic acid and its compounds, ceramides and their homologues, compounds of plants such as camomile, bearberry, the aloe (vera, ferox, bardensis) family, mulberry and skullcap; a kiwi fruit juice (Actinidia chinensis) sold by Gattefosse, an extract of Paeonia suffructicosa root such as that sold by Ichimaru Pharcos under the name BOTANPI LIQUID B®, an extract of brown sugar (Saccharum officinarum), such as the extract of molasses sold by Taiyo Kagaku under the name MOLASSES LIQUID, without this list being exhaustive.

As preferred depigmenting agents, use will be made of vitamin C and its compounds and especially vitamin CG, vitamin CP and 3-O-ethyl-vitamin C, alpha- and beta-arbutin, ferulic acid, kojic acid, resorcinol and its compounds, calcium D-pantetheine sulphonate, lipoic acid, ellagic acid, vitamin B3, a kiwi fruit juice (Actinidia chinensis) sold by Gattefossé, an extract of Paeonia suffruticosa root such as that sold by Ichimaru Pharcos under the name BOTANPI LIQUID B®.

### 5. Antioxidants

Mention may especially be made of tocopherol and esters thereof, in particular tocopherol acetate; ascorbic acid and its compounds, in particular magnesium ascorbyl phosphate and ascorbyl glucoside; ferulic acid; serine; ellagic acid, phloretin, polyphenols, tannins, tannic acid, epigallocatechins and natural extracts containing them, anthocyans, rosemary extracts, olive leaf extracts such as those from Silab, green tea extracts, resveratrol and its compounds, ergothioneine, N-acetylcysteine, an extract of the brown alga Pelvetia canaliculata such as PELVETIANE® from Secma, chlorogenic acid, biotin, chelating agents, such as BHT, BHA, N,N′-bis(3,4,5-trimethoxybenzyl)-ethylenediamine and salts thereof; idebenone, plant extracts such as PRONALEN BIOPROTECT™ from Provital; coenzyme Q10, bioflavonoids, SODs, phytantriol, lignans, melatonin, pidolates, glutathione, caprylyl glycol, phloretin, TOTAROL™ or extract of Podocarpus totara containing totarol (totara-8,11,13-trienol or 4b,5,6,7,8,8a,9,10-octahydro-4-b,8,8-trimethyl-1-(1-methylethyl)-2-phenanthrenol; a jasmine extract such as that sold by Silab under the name HELISUN®; hesperitin laurate such as FLAVAGRUM PEG® by Engelhard Lyon; an extract of Paeonia sufftruticosa root such as that sold by Ichimaru Pharcos under the name BOTANPI LIQUID B®; a lychee extract such as the extract of lychee pericarp sold by Cognis under the name LITCHIDERM LS 9704®, an extract of pomegranate fruit (Punica granatum), such as that sold by Draco Natural Products.

As other anti-ageing agents, mention may be made of DHEA and its compounds, boswellic acid, rosemary extracts, carotenoids (β-carotene, zeaxanthin, lutein), cysteic acid, copper compounds and jasmonic acid.

As preferred antioxidants, use will especially be made of ferulic acid; serine; phloretin, a pomegranate extract, biotin, chelating agents such as BHT, BHA, N,N′-bis(3,4,5-trimethoxybenzyl)ethylenediamine and its salts, caprylyl glycol, phloretin, TOTAROL™, a jasmine extract such as that sold by Silab under the name HELISUN®; hesperetin laurate such as FLAVAGRUM PEG from Engelhard Lyon; an extract of Paeonia suffruticosa root such as that sold by Ichimaru Pharcos under the name BOTANPI LIQUID B®.

### 6. Dermo-Relaxing or Dermo-Decontracting Agents

Mention may be made, as examples, of manganese gluconate and other salts, adenosine, alverine citrate and its salts, glycine, an extract of Iris pallida, a hexapeptide (ARGERILINE R from Lipotec) or sapogenins such as wild yam and the carbonylated amines described in Application EP 1 484 052. As an example of sapogenins, mention may be made of those described in Patent Application WO 02/47650, in particular wild yam, the diosgenin extracted, in particular, from Dioscorea opposita or any extract naturally containing, or containing after treatment, one or more sapogenins (wild yam rhizome, agave leaf which contains hecogenin and tigogenin, extract of Liliacea and more particularly yucca or smilax containing smilagenin and sarsapogenin, or sarsaparilla root) or ACTIGEN Y from Actives Organics; or ginger.

Mention may also be made of DMAE (dimethyl MEA), extracts of sea fennel, of rock rose, of helichrysum, of aniseed, of paracress, and an extract of Acmella oleracea such as GATULINE® EXPRESSION from Gattefossé.

As preferred dermo-relaxing agents, mention will be made of adenosine, manganese gluconate, wild yam, sea fennel, glycine and alverine.

### 7. Anti-Glycation Agents

The term “anti-glycation agent” is understood to mean a compound that prevents and/or reduces the glycation of skin proteins, in particular of dermal proteins such as collagen.

As anti-glycation agents, mention may especially be made of the plant extracts of the Ericaceae family, such as an extract of blueberry (Vaccinium angustifolium or Vaccinium myrtillus), for example that sold under the name BLUEBERRY HERBASOL EXTRACT PG by Cosmetochem, ergothioneine and compounds thereof, hydroxystilbenes and compounds thereof, such as resveratrol and 3,3′,5,5′-tetrahydroxystilbene (these anti-glycation agents are described in Applications FR 2 802 425, FR 2 810 548, FR 2 796 278 and FR 2 802 420 respectively), dihydroxystilbenes and compounds thereof, polypeptides of arginine and of lysine such as that sold under the name AMADORINE® by Solabia, carcinine hydrochloride (sold by Exsymol under the name ALISTIN®), an extract of Helianthus annuus such as ANTIGLYSKIN® from Silab, wine extracts such as the extract of powdered white wine on a maltodextrin support sold under the name VIN BLANC DESHYDRATE 2F by Givaudan, thioctic acid (or α-lipoic acid), a mixture of extract of bearberry and of marine glycogen such as AGLYCAL LS 8777® from Laboratoires Seriobiologiques, an extract of black tea such as KOMBUCHKA® from Sederma and mixtures thereof.

As preferred anti-glycation agents, mention will be made of blueberry extracts (Vaccinium myrtillus) and the extract of black tea.

8. Agents that Stimulate the Synthesis of Dermal and/or Epidermal Macromolecules and/or that Prevent their Degradation:

Among the active agents that stimulate the dermal macromolecules or that prevent their degradation, mention may be made of those which act:
either on the collagen synthesis, such as extracts of Centella asiatica, asiaticosides and compounds; ascorbic acid or vitamin C and compounds thereof; synthetic peptides such as iamin, biopeptide CL or palmitoyl oligopeptide sold by Sederma; peptides extracted from plants, such as the soybean hydrolysate sold by Coletica under the trade name PHYTOKINE®; rice peptides such as NUTRIPEPTIDE® by Silab, methylsilanol mannuronate such as ALGISIUM C® sold by Exsymol; plant hormones such as auxins and lignans; folic acid; and an extract of Medicago sativa (alfalfa) such as that sold by Silab under the name VITANOL®; a peptide extract of hazelnut such as that sold by Solabia under the name NUTELINE C®; and arginine;
   or on the inhibition of collagen degradation, in particular agents that act on the inhibition of metalloproteinases (MMPs) such as, more particularly, MMP 1, 2, 3 and 9. Mention may be made of: retinoids and compounds, extracts of Medicago sativa such as VITANOL® from Silab, an extract of Aphanizomenon flos-aquae (cyanophyceae) sold under the name LANABLUE® by Atrium Biotechnologies, oligopeptides and lipopeptides, lipoamino acids, the malt extract sold by Coletica under the trade name COLLALIFT®; blueberry or rosemary extract; lycopene; isoflavones, their compounds or the plant extracts containing them, in particular the extracts of soybean (sold, for example, by Ichimaru Pharcos under the trade name FLAVOSTERONE SB®), of red clover, of flax, of kakkon; a lychee extract such as the extract of lychee pericarp sold by Cognis under the name LITCHIDERM LS 9704®; dipalmitoyl hydroxyproline sold by Seppic under the name SEPILIFT DPHP®: Baccharis genistelloides or BACCHARINE sold by Silab, an extract of moringa such as ARGANYL LS 9781®) by Cognis; the sage extract described in Application FR-A-2812544 from the Labiatae family (Salvia officinalis from Flacksmann), a rhododendron extract, blueberry extract, an extract of Vaccinium myrtillus such as those described in application FR-A-2814950.
or on the synthesis of molecules belonging to the family of elastins (elastin and fibrillin) such as: retinol and compounds, in particular retinol palmitate; extract of Saccharomyces cerivisiae sold by LSN under the trade name CYTOVITIN®; and the extract of Macrocystis pyrifera alga sold by Secma under the trade name KELPADELIE®; a peptide extract of hazelnut such as that sold by Solabia under the name NUTELINE C®;
or on the inhibition of the degradation of elastin such as the peptide extract of Pisum sativum seeds sold by LSN under the trade name PARELASTYL®; heparinoids; and the N-acylamino acid compounds described in Application WO 01/94381 such as {2-[acetyl-(3-trifluoromethylphenyl)amino]-3-methylbutyrylamino}-acetic acid, also known as N—[N-acetyl-N′-(3-trifluoromethyl)phenylvalyl]glycine or N-acetyl-N-[3-(trifluoromethyl)phenyl]valylglycine or acetyl trifluoromethyl phenyl valylglycine, or an ester of the latter with a C₁-C₆ alcohol; an extract of rice peptides such as COLHIBIN® by Pentapharm, or an extract of Phyllanthus emblica such as EMBLICA® from Rona;
or on the synthesis of glycosaminoglycans, such as the product of fermentation of milk with Lactobacillus vulgaris, sold by Brooks under the trade name BIOMIN YOGOURTH®; the extract of brown alga Padina pavonica sold by Alban Müller under the trade name HSP3®; the extract of Saccharomyces cerevisiae especially available from Silab under the trade name FIRMALIFT® or from LSN under the trade name CYTOVITIN®; an extract of Laminaria ochroleuca such as LAMINAINE® by Secma; essence of Mamaku from Lucas Meyer, a cress extract (ODRALINE® from Silab);
or on the synthesis of fibronectin, such as the extract of the zooplankton Salina sold by Seporga under the trade name GP4G®; the yeast extract especially available from Alban Müller under the trade name DRIELINE®; and the palmitoyl pentapeptide sold by Sederma under the trade name MATRIXIL®.

Among the active agents that stimulate the epidermal macromolecules, such as fillagrin and keratins, mention may especially be made of the lupin extract sold by Silab under the trade name STRUCTURINE®; the extract of Fagus sylvatica beech buds sold by Gattefossé under the trade name GATULINE® RC; and the extract of zooplankton Salina sold by Seporga under the trade name GP4G®; copper tripeptide from Procyte; a peptide extract of Voandzeia subterranea such as that sold by Laboratoires Sérobiologiques under the trade name FILLADYN LS 9397®.

Preferably, an active agent will be used that stimulates the synthesis of dermal and/or epidermal macromolecules and/or that prevent their degradation, chosen from agents that stimulate the synthesis of glycosaminoglycans, agents that inhibit the degradation of elastin, agents that stimulate the synthesis of fibronectin, agents that stimulate the synthesis of epidermal macromolecules, and mixtures thereof.

Even more preferably, an active agent will be used that stimulates the synthesis of glycosaminoglycans chosen from an extract of brown alga Padina pavonica, an extract of Saccharomyces cerevisiae, an extract of Laminaria ochroleuca, essence of Mamaku, a cress extract and mixtures thereof.

As preferred active agents that stimulate the synthesis of dermal and/or epidermal macromolecules and/or that prevent their degradation, mention may be made of:

synthetic peptides such as iamin, biopeptide CL or palmitoyl oligopeptide sold by Sederma; peptides extracted from plants, such as the soybean hydrolysate sold by Coletica under the trade name PHYTOKINE®; rice peptides such as NUTRIPEPTIDE® from Silab, methylsilanol mannuronate such as ALGISIUM C® sold by Exsymol; folic acid; and an extract of Medicago sativa (alfalfa) such as that sold by Silab under the name VITANOL®; a peptide extract of hazelnut such as that sold by Solabia under the name NUTELINE C®; arginine; an extract of Aphanizomenon flos-aquae (cyanophyceae) sold under the name LANABLUE® by Atrium Biotechnologies, the malt extract sold by Coletica under the trade name COLLALIFT®; lycopene; a lychee extract; an extract of moringa such as ARGANYL LS 9781® from Cognis; an extract of Vaccinium myrtillus such as those described in Application FR-A-2814950; retinol and compounds, in particular retinol palmitate; the extract of Saccharomyces cerevisiae sold by LSN under the trade name CYTOVITIN®; a peptide extract of hazelnut such as that sold by Solabia under the name NUTELINE C®; {2-[acetyl-(3-trifluoromethyl-phenyl)amino]-3-methylbutyrylamino}acetic acid, also known as N—[N-acetyl-N′-(3-trifluoromethyl)-phenylvalyl]glycine or N-acetyl-N-[3-(trifluoromethyl)-phenyl]valylglycine or acetyl trifluoromethyl phenyl valylglycine, or an ester of the latter with a C₁-C₆ alcohol; an extract of rice peptides such as COLHIBIN® from Pentapharm, or an extract of Phyllanthus emblica such as EMBLICA® from Rona; the extract of brown alga Padina pavonica sold by Alban Müller under the trade name HSP3®; the extract of Saccharomyces cerevisiae available especially from Silab under the trade name FIRMALIFT® or from LSN under the trade name CYTOVITIN®; an extract of Laminaria ochroleuca such as LAMINAINE® from Secma; essence of Mamaku from Lucas Meyer, the lupin extract sold by Silab under the trade name STRUCTURINE®; the extract of Fagus sylvatica beech buds sold by Gattefosse under the trade name GATULINE® RC.
9. Agents that Stimulate the Proliferation of Fibro-Blasts or of Keratinocytes and/or the Differentiation of Keratinocytes

The agents that stimulate the proliferation of fibro-blasts that can be used in the composition according to the invention may, for example, be chosen from plant proteins or polypeptides, extracted especially from soybean (for example a soybean extract sold by LSN under the name ELESERYL SH-VEG 8® or sold by Silab under the trade name RAFFERMINE®); an extract of hydrolysed soybean proteins such as RIDULISSE® from Silab; and plant hormones such as gibberellins and cytokinins; a peptide extract of hazelnut such as that sold by Solabia under the name NUTELINE C®.

Preferably, an agent will be used that favours the proliferation and/or differentiation of keratinocytes.

The agents that stimulate the proliferation of keratinocytes, that can be used in the composition according to the invention, comprise in particular adenosine; phloroglucinol, the extract of Hydrangea macrophylla leaf such as AMACHA LIQUID E® from Ichimaru Pharcos, a yeast extract such as STIMODERM® from CLR; the extract of Larrea divaricata such as CAPISLOW® from Sederma, mixtures of extracts of papaya, of olive leaves and of lemon such as XYLEINE® from Vincience, the extract of Hydrangea macrophylla leaf such as AMACHA LIQUID E® from Ichimaru Pharcos, retinol and its esters including retinyl palmitate, phloroglucinol, the walnut meal extracts sold by Gattefossé and the extracts of Solanum tuberosum such as DERMOLECTINE® sold by Sederma.

Among the agents that stimulate the differentiation of the keratinocytes are, for example, minerals such as calcium; sea fennel, a peptide extract of lupin such as that sold by Silab under the trade name STRUCTURINE®; sodium beta-sitosteryl sulphate such as that sold by Seporga under the trade name PHYTOCOHESINE®; and a water-soluble extract of maize such as that sold by Solabia under the trade name PHYTOVITYL®; a peptide extract of Voandzeia subterranea such as that sold by Laboratoires Sérobiologiques under the trade name FILLADYN LS 9397®; and lignans such as secoisolariciresinol, retinol and its esters including retinyl palmitate.

Among agents that stimulate the proliferation and/or the differentiation of keratinocytes, mention may also be made of oestrogens such as oestradiol and homologues; cytokines.

As preferred active agents that stimulate proliferation of fibroblasts or keratinocytes and/or the differentiation of keratinocytes, mention will be made of plant proteins or polypeptides, extracted especially from soybean (for example a soybean extract sold by LSN under the name ELESERYL SH-VEG 8® or sold by Silab under the trade name RAFFERMINE®); an extract of hydrolysed soybean proteins such as RIDULISSE® from Silab; a peptide extract of hazelnut such as that sold by Solabia under the name NUTELINE C®; adenosine; phloroglucinol, a yeast extract such as STIMODERM® from CLR; a peptide extract of lupin such as that sold by Silab under the trade name STRUCTURINE®; a water-soluble extract of maize such as that sold by Solabia under the trade name PHYTOVITYL®; a peptide extract of Voandzeia subterranea such as that sold by Laboratoires Sérobiologiques under the trade name FILLADYN LS 9397®; retinol and its esters including retinyl palmitate.

10. Agents that Favour Maturing of the Horny Envelope

It will be possible to use, in the compositions of the invention, agents that are involved in maturing of the horny envelope which deteriorates with age and leads to a reduction of transglutaminase activity. Mention may be made, for example, of urea and its compounds and in particular HYDROVANCE® from National Starch and the other active agents mentioned in L′Oreal Application FR 2 877 220 (unpublished).

### 11. NO-Synthase Inhibitors

The agent having an NO-synthase inhibitor action may be chosen from PCOs (procyanidolic oligomers); plant extracts of the species Vitis vinifera especially sold by Euromed under the name LEUCOCYANIDINES DE RAISINS EXTRA [grape leucocyanidins], or else by Indena under the name LEUCOSELECT®, or finally by Hansen under the name EXTRAIT DE MARC DE RAISIN [grape-cake extract]; plant extracts of the species Olea europaea preferably obtained from olive leaves and especially sold by Vinyals in the form of a dry extract, or by Biologia & Technologia under the trade name EUROL® BT; plant extracts of the species Gingko biloba preferably a dry aqueous extract of this plant sold by Beaufour under the trade name GINKGO BILOBA EXTRAIT STANDARD and mixtures thereof.

### 12. Peripheral Benzodiazepine Receptor (PBR) Antagonists

Mention may be made, for example, of 1-(2-chloro-phenyl)-—N-(1-methylpropyl)-3-isoquinolinecarboxamide; the compounds described in Applications WO 03/030937, WO 03/068753, and pyridazino[4,5-b]indole-1-acetamide compounds of general formula (VII) as described in document WO 00/44384.

### 13. Agents Increasing the Activity of the Sebaceous Gland

Mention may be made, for example, of methyl dihydro-jasmonate, hecogenin, hedione, O-linoleyl-6-D-glucose and mixtures thereof.

### 14. Agents Stimulating Cell Energy Metabolism

The active agent that stimulates the energy metabolism of cells may for example be chosen from among biotin, an extract of Saccharomyces cerevisiae such as PHOSPHOVITAL® from Sederma, the mixture of sodium, manganese, zinc and magnesium salts of pyrrolidone carboxylic acid such as PHYSIOGENYL® from Solabia, a mixture of zinc, copper and magnesium gluconate such as SEPITONIC M3® from Seppic, and mixtures thereof; a beta-glucan derived from Saccharomyces cerevisiae such as that sold by Mibelle AG Biochemistry.

### 15. Tightening Agents

The term “tightening agent” which can be used according to the invention, is understood to mean compounds capable of having a tightening effect, that is to say which can tighten the skin.

Generally, the term “tightening agent” is understood, according to the invention, to mean any compounds that are soluble or dispersible in water at a temperature ranging from 25° C. to 50° C., at the concentration of 7% by weight in water or at the maximum concentration at which they form a medium of homogeneous appearance, and that produce at this concentration of 7%, or at this maximum concentration in the water, a shrinkage of more than 15% in the test described below.

The maximum concentration at which they form a medium of homogeneous appearance is determined to within ±10% and preferably to within ±5%.

The expression “medium of homogeneous appearance” is understood to mean a medium that does not have any aggregates visible to the naked eye.

In order to determine said maximum concentration, the tightening agent is gradually added to the water, with stirring using a deflocculator, at a temperature ranging from 25° C. to 50° C., then the mixture continues to be stirred for one hour. Next, after 24 hours, it is observed whether the mixture thus prepared has a homogeneous appearance (absence of aggregates visible to the naked eye).

The tightening effect may be characterized by an in vitro shrinkage test.

As described previously, a homogenous mixture of the tightening agent in water is prepared first, at the concentration of 7% by weight or at the maximum concentration defined previously.

30 μl of the homogeneous mixture is deposited on a rectangular elastomeric test piece (10×40 mm, therefore having an initial width Lₒ of 10 mm) having a modulus of elasticity of 20 MPa and a thickness of 100 μm.

After drying for 3 h at 22±3° C. and 40±10% relative humidity RH, the elastomeric test piece has a shrunken width, denoted by L₃ₕ due to the tension exerted by the deposited tightening agent.

The tightening effect (TE) of said agent is then quantified in the following manner:

‘TE’=(L₀−L₃ₕ/L₀)×100 as % with L₀=initial width 10 mm and L₃ₕ=width after drying for 3 h

The tightening agent may be chosen from:

a) plant or animal proteins and their hydrolysates;
b) polysaccharides of natural origin;
c) mixed silicates;
d) colloidal particles of inorganic fillers;
e) synthetic polymers;
and mixtures thereof.

A person skilled in the art will know how to choose, from the chemical categories listed above, the materials that fulfil the tightening test such as described previously.

Mention may especially be made of:
(a) proteins and hydrolysates of plant proteins, in particular of maize, rye, wheat, buckwheat, sesame, spelt, pea, bean, lentil, soybean and lupin,
(b) polysaccharides of natural origin, especially (a) polyholosides, for example (i) in the form of starch derived especially from rice, maize, potato, cassava, peas, wheat, oats, etc. or (ii) in the form of carrageenans, alginates, agars, gellans, cellulose polymers and pectins, advantageously as an aqueous dispersion of gel microparticles, and (b) latices composed of shellac resin, gum sandarac, dammars, elemis, copals, cellulose compounds, and mixtures thereof;
(c) mixed silicates, especially phyllosilicates and in particular laponites;
(d) colloidal particles of inorganic filler having a number-average diameter between 0.1 and 100 nm, preferably between 3 and 30 nm, and chosen, for example, from: silica, silica/alumina composites, cerium oxide, zirconium oxide, alumina, calcium carbonate, barium sulphate, calcium sulphate, zinc oxide and titanium dioxide. As composite silica/alumina colloidal particles that can be used in the compositions according to the invention, mention may be made, for example, of those sold by Grace under the names LUDOX AM, LUDOX AM-X 6021, LUDOX HSA and LUDOX TMA.
(e) synthetic polymers, such as polyurethane latices or acrylic/silicone latices, in particular those described in Patent Application EP 1 038 519, such as a propylthio(polymethyl acrylate), propylthio(polymethyl methacrylate) and propylthio(polymethacrylic acid) grafted polydimethylsiloxane or else a propyl-thio(polyisobutyl methacrylate) and propylthio(poly-methacrylic acid) grafted polydimethylsiloxane. Such grafted silicone polymers are especially sold by 3M under the trade names VS 80, VS 70 and L021.

The tightening agent will be present in the composition in an effective amount to obtain the desired biological effect according to the invention.

By way of example, the tightening agent may be included in the composition according to the invention in an amount ranging from 0.01 to 30% by weight of active material, preferably from 1% to 30% by weight of active material, relative to the total weight of the composition.

The expression “active material” is understood to exclude the medium in which the tightening agent is optionally dissolved or dispersed in its commercial form, for example in the case of dispersions of colloidal particles.

It is also possible, especially for complementing and/or increasing the efficacy of the effect of the tightening agents, to use agents that increase the expression of mechanoreceptors, such as agents that increase the expression of integrins.

By way of example, mention may be made of a rye seed extract, such as that sold by Silab under the name COHELISS®.

### 16. Lipo-Restructuring Agents

The expression “lipo-restructuring agents” is understood, according to the invention, to mean agents capable of stimulating lipogenesis and favouring adipocyte differentiation, thus making it possible to prevent or slow down the wasting of fat contained in the skin-supporting tissues, otherwise known as “wasting of the fat structure of the skin”.

The expression “fat structure of the skin” is understood to mean the network of fat cells which form the volumes on which the skin of the face rests and moulds itself to.

These agents are intended for
decreasing the loss of skin density and/or the wasting of the fat structure of the skin, in particular on the cheeks and in the area around the eye; and/or
preventing the collapse and/or hollowing of the facial volumes, the loss of consistency of the skin and/or its maintenance, in particular on the cheeks and in the area around the eye; and/or
improving the underlying volumes of the skin of the face and/or of the neck, in particular on the cheeks, the oval of the face and in the area around the eye; and/or
improving the density, the springiness and maintenance of the skin, in particular on the cheeks, the oval of the face and in the area around the eye; and/or
reshaping the facial features, in particular the oval of the face.

As examples of lipo-restructuring agents, mention may especially be made of an extract of black tea, such as the extract of fermented black tea sold by Sederma under the name KOMBUCHKA®, and an extract of Artemisia abrotanum, such as that sold by Silab under the name PULPACTYL®.

### 17. Slimming Agents

As slimming (lipolytic) agents, mention may especially be made of caffeine, theophylline, and its compounds, theobromine, sericosine, Asiatic acid, acefylline, aminophylline, chloroethyltheophylline, diprofylline, diniprophylline, etamiphylline and its compounds, etofylline, proxyphylline; extracts of tea, coffee, guarana, mate, cola (Cola nitida) and especially the dry extract of guarana fruit (Paulina sorbilis) containing 8 to 10% of caffeine; extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St. John's wort (Hypericum perforatum), of butcher's broom (Ruscus aculeatus L), of meadowsweet (Filipendula ulmaria L), of orthosiphon (Orthosiphon stamincus benth), of birch (Betula alba), of cecropia and of argan tree; extracts of Ginkgo biloba, extracts of horsetail, extracts of escin, extracts of cangzhu, extracts of Chrysanthellum indicum, extracts of Dioscorea plants rich in diosgenin or pure diosgenin or hecogenin and compounds thereof, extracts of Ballota, extracts of Guioa, of Davallia, of Terminalia, of Barringtonia, of Trema, of Antirobia, extract of bitter orange petit grain; an extract of cocoa bean shells (Theobroma cacao) such as that sold by Solabia under the name CAOBROMINE®.

### 18. Agents Favouring Skin Microcirculation

The active agent that acts on skin microcirculation may be used to prevent dulling of the complexion and/or improve the appearance of the area around the eye, in particular to reduce dark circles. It may be chosen, for example, from an extract of maritime pine bark such as PYCNOGENOL from Biolandes, manganese gluconate (GIVOBIO GMn® from Seppic), an extract of Ammi visnaga such as VISNADINE from Indena, extract of lupin (ECLALINE® from Silab), the coupling of hydrolysed wheat protein/palmitic acid with palmitic acid such as EPALINE 100 from Laboratoires Cariléne, the extract of bitter orange flower (REMODULINE® from Silab), vitamin P and its compounds such as 4-methylesculetol sodium monoethanoate sold under the name PERMETHOL® by Sephytali, extracts of Ruscus, of horse chestnut, of ivy, of ginseng and of melilot, caffeine, nicotinate and its compounds, lysine and its compounds such as ASPARLYNE® from Solabia, an extract of black tea such as KOMBUCHKA from Sederma; rutin salts: an extract of Corallina officinalis alga such as that sold by Codif; and mixtures thereof.

As preferred agents that favour skin microcirculation, mention will be made of caffeine, an extract of bitter orange flower, an extract of black tea, rutin salts, an extract of Corallina officinalis alga.

### 19. Soothing or Anti-Irritant Agents

The term “soothing agent” is understood to mean a compound that makes it possible to reduce the sensation of stinging, itching or tautness of the skin.

As soothing agents that can be used in the composition according to the invention, mention may be made of: procyanidolic oligomers, vitamins E, C, B5 and B3, caffeine and its compounds, pentacyclic triterpenes and the plant extracts containing them, β-glycyrrhetinic acid and its salt or compounds (stearyl glycyrrhetate, 3-stearoyloxy glycyrrhetic acid, glycyrrhetinic acid monoglucuronide) and also the plants containing them (e.g. Glycyrrhiza glabra), oleanolic acid and its salts, ursolic acid and its salts, boswellic acid and its salts, betulinic acid and its salts, an extract of Paeonia suffruticosa and/or lactiflora, an extract of Laminaria saccharina, extracts of Centella asiatica, Canola oil, bisabolol, the phosphoric diester of vitamin E and C such as SEPIVITAL EPC® from Seppic, camomile extracts, allantoin, unsaturated omega-3 oils such as musk rose oil, blackcurrant oil, Ecchium oil, fish oil, calophyllum oil, plankton extracts, capryloyl glycine, a mixture of water lily flower extract and of palmitoyl proline such as that sold under the name SEPPICALM VG® by Seppic, an extract of Boswellia serrata, an extract of Centipeda cunninghami such as that sold under the name CEHAMI PF® by TRI-K Industries, an extract of sunflower seeds in particular HELIOXINE® from Silab, an extract of Linum usitatissimum seeds such as SENSILINE® from Silab, tocotrienols, piperonal, an extract of Epilobium angustifolium such as that sold under the name CANADIAN WILLOWHERB EXTRACT by Fytokem Products, aloe vera, phytosterols, cornflower water, rose water, an extract of mint, in particular mint leaves such as CALMISKIN® by Silab, aniseed compounds, filamentous bacteria such as Vitreoscilla filiformis such as described in Patent EP 761 204 and sold by Chimex under the name MEXORYL SBG®, an extract of rose petals such as ROSE FLOWER HERBASOL® EXTRACT from Cosmetochem, shea butter, a mixture of the wax fraction of barley seed obtained by supercritical CO₂, shea butter and argan oil such as STIMUTEX AS® from Pentapharm, alkaline-earth metal salts especially strontium salts, a fermented extract of Alteromonas sold under the name ABYSSINE® by Atrium Biotechnologies; spring waters from the Vichy basin, such as the waters originating from the Célestins, Chomel, Grande-Grille, Hôpital, Lucas and Parc sources, and preferably water from the Lucas source; an extract of Eperua falcata bark such as that sold by Cognis under the name EPERULINE®; an extract of Paeonia suffruticosa root such as that sold by Ichimaru Pharcos under the name BOTANPI LIQUID B®; and mixtures thereof.

As preferred soothing agents according to the invention, use will be made of:

β-glycyrrhetinic acid and its salts or compounds (stearyl glycyrrhetate, 3-stearoyloxy glycyrrhetic acid, glycyrrhetinic acid monoglucuronide) and also the plants containing them (e.g. Glycyrrhiza glabra); ursolic acid and its salts; extracts of Centella asiatica, Canola oil, bisabolol; camomile extracts, allantoin; a mixture of water lily flower extract and of palmitoyl proline such as that sold under the name SEPPICALM VG® by Seppic; aloe vera, rose water, an extract of mint, in particular mint leaves such as CALMISKIN® from Silab, filamentous bacteria such as Vitreoscilla filiformis such as described in Patent EP 761 204 and sold by Chimex under the name MEXORYL SBG®, an extract of rose petals such as ROSE FLOWER HERBASOL®EXTRACT from Cosmetochem, shea butter, a fermented extract of Alteromonas sold under the name ABYSSINE® by Atrium Biotechnologies; spring waters from the Vichy basin, such as the waters originating from the Célestins, Chomel, Grande-Grille, Hôpital, Lucas and Parc sources, and preferably water from the Lucas source; an extract of Eperua falcata bark such as that sold by Cognis under the name EPERULINE®; an extract of Paeonia suffruticosa root such as that sold by Ichimaru Pharcos under the name BOTANPI LIQUID B®; and mixtures thereof.

### 20. Sebum-Regulating or Antiseborrhoeic Agents

The term “sebum-regulating or antiseborrhoeic” agents is understood to mean especially agents capable of regulating the activity of the sebaceous glands.

Mention may especially be made of:
retinoic acid, benzoyl peroxide, sulphur, vitamin B6 (or pyridoxine), selenium chloride, and sea fennel;
mixtures of extract of cinnamon, tea and octanoyl glycine such as SEPICONTROL A5 TEA® from Seppic;
the mixture of cinnamon, sarcosine and octanoyl glycine sold especially by Seppic under the trade name SEPICONTROL A5®;
zinc salts such as zinc gluconate, zinc pyrrolidone carboxylate (or zinc pidolate), zinc lactate, zinc aspartate, zinc carboxylate, zinc salicylate and zinc cysteate;
copper compounds and in particular copper pidolate such as CUIVRIDONE® from Solabia;
plant extracts of the species Arnica montana, Cinchona succirubra, Eugenia caryophyllata, Humulus lupulus, Hypericum perforatum, Mentha piperita, Rosmarinus officinalis, Salvia officinalis and Thymus vulgaris, all sold, for example, by Maruzen;
extracts of meadowsweet (Spiraea ulamaria) such as that sold under the name SEBONORMINE® by Silab;
extracts of the Laminaria saccharina algae such as that sold under the name PHLOROGINE® by Biotechmarine;
mixtures of extracts of burnet (Sanguisorba officinalis/Poterium officinale) roots, ginger (Zingiber officinalis) rhizomes and cinnamon (Cinnamomum cassia) bark such as that sold under the name SEBUSTOP® by Solabia;
linseed extracts such as that sold under the name LINUMINE® by Lucas Meyer;
Phellodendron extracts such as those sold under the name PHELLODENDRON EXTRACT BG by Maruzen or OUBAKU LIQUID B by Ichimaru Pharcos;
mixtures of argan oil, saw palmetto extract and sesame seed extract such as that sold under the name REGU SEB® by Pentapharm;
mixtures of extracts of rosebay willow herb, Terminalia chebula, nasturtium and bioavailable zinc (microalgae) such as that sold under the name SEBORILYS® by Greentech;
extracts of Pygeum africanum such as that sold under the name PYGEUM AFRICANUM STEROLIC LIPID EXTRACT by Euromed;
extracts of saw palmetto such as those sold under the name VIAPURE SABAL by Actives International, or those sold by Euromed;
mixtures of extracts of plantain, Berberis aquifolium and sodium salicylate such as that sold under the name SEBOCLEAR® by Rahn;
clove extract such as that sold under the name CLOVE EXTRACT POWDER by Maruzen;
argan oil such as that sold under the name LIPOFRUCTYL® by Laboratoires Sérobiologiques;
lactic protein filtrates such as that sold under the name NORMASEB® by Sederma;
extracts of Laminaria algae, such as that sold under the name LAMINARGHANE® by Biotechmarine;
oligosaccharides of Laminaria digitata algae such as that sold under the name PHYCOSACCHARIDE AC by Codif;
sugar cane extracts, such as that sold under the name POLICASONOL® by Sabinsa;
sulphonated shale oil, such as that sold under the name ICHTYOL PALE® by Ichthyol;
meadowsweet (Spiraea ulmaria) extracts, such as that sold under the name CYTOBIOL® ULMAIRE by Libiol;
sebacic acid, especially sold in the form of a sodium polyacrylate gel under the name SEBOSOFT® by Sederma;
glucomannans extracted from konjac tuber and modified by alkylsulphonate chains, such as that sold under the name BIOPOL BETA by Arch Chemical;
extracts of Sophora angustifolia, such as those sold under the name SOPHORA POWDER or SOPHORA EXTRACT by Bioland;
extracts of Cinchona succirubra bark such as that sold under the name RED BARK HS by Alban Muller;
extracts of Quillaja saponaria such as that sold under the name PANAMA WOOD HS by Alban Muller;
glycine grafted onto an undecylene chain, such as that sold under the name LIPACIDE UG OR by Seppic;
mixture of oleanolic acid and nordihydroguaiaretic, such as that sold in the form of a gel under the name AC.NET by Sederma;
phthalimidoperoxyhexanoic acid;
trialkyl (C₁₂-C₁₃) citrate sold under the name COSMACOL® ECI by Sasol; trialkyl (C₁₄-C₁₅) citrate sold under the name COSMACOL® ECL by Sasol;
10-hydroxydecanoic acid, and especially mixtures of 10-hydroxydecanoic acid, sebacic acid and 1,10-decanediol such as that sold under the name ACNACIDOL® BG by Vincience; and
mixtures thereof.

As preferred antiseborrhoeic active agents, mention may be made of:
benzoyl peroxide and vitamin B6 (or pyridoxine);
zinc salts such as zinc gluconate, zinc pyrrolidonecarboxylate (or zinc pidolate), zinc lactate, zinc aspartate, zinc carboxylate, zinc salicylate and zinc cysteate;
meadowsweet (Spiraea ulmaria) extracts, such as that sold under the name SEBONORMINE® by Silab;
extracts of the alga Laminaria saccharina, such as that sold under the name PHLOROGINE® by Biotechmarine;
mixtures of extracts of salad burnet root (Sanguisorba officinalis/Poterium officinale), of ginger rhizomes (Zingiber officinalis) and of cinnamon bark (Cinnamomum cassia), such as that sold under the name SEBUSTOP™ by Solabia;
clove extract, such as that sold under the name CLOVE EXTRACT POWDER by Maruzen;
lactic protein filtrates, such as that sold under the name NORMASEB® by Sederma;
European meadowsweet (Spiraea ulmaria) extracts, such as that sold under the name CYTOBIOL® ULMAIRE by Libiol;
sebacic acid, especially sold in the form of a sodium polyacrylate gel under the name SEBOSOFT® by Sederma;
glycine grafted onto an undecylenic chain, such as that sold under the name LIPACIDE UG OR by SEPPIC;
tri(C₁₂-C₁₃)alkyl citrate sold under the name COSMACOL® ECI by the company Sasol; tri(C₁₄-C₁₅)alkyl citrate sold under the name COSMACOL® ECL by Sasol;
10-hydroxydecanoic acid, and especially mixtures of 10-hydroxydecanoic acid, of sebacic acid and of 1,10-decanediol, such as that sold under the name ACNACIDOL® BG by Vincience; and
mixtures thereof.

Preferentially, the antiseborrhoeic active agent is chosen from:
zinc salts such as zinc gluconate, zinc pyrrolidonecarboxylate (or zinc pidolate), zinc lactate, zinc aspartate, zinc carboxylate, zinc salicylate and zinc cysteate; and preferably zinc pyrrolidonecarboxylate (or zinc pidolate) or zinc salicylate;
clove extract, such as that sold under the name CLOVE EXTRACT POWDER by Maruzen;
glycine grafted onto an undecylenic chain, such as that sold under the name LIPACIDE UG OR by SEPPIC;
tri(C₁₂-C₁₃)alkyl citrate sold under the name COSMACOL® ECI by Sasol; tri(C₁₄-C₁₅)alkyl citrate sold under the name COSMACOL® ECL by Sasol;
and mixtures thereof.

The antiseborrhoeic active agent is, for example, present in a content ranging from 0.1% to 10% by weight, preferably from 0.1% to 5% by weight and preferentially from 0.5% to 3% by weight relative to the total weight of the composition.

### 21. Astringents

According to the invention, the term “astringents” means agents for combating the dilation of the sebaceous follicles.

As astringents that may be used in the composition according to the invention, mention may be made of extracts of mushroom pulp (Polyporus officinalis), for instance LARICYL LS8865® from Cognis, extracts of Terminalia catappa and Sambucus nigra, for instance PHYTOFIRM LS9120® from Cognis, extracts of gall nut, for instance TANLEX VE® from Ichimaru Pharcos, aluminium hydroxychloride, centella extracts (e.g. PLANTACTIV CENTELLA from Cognis), dicetyl dimethylammonium chloride, for instance VARISOFT 432 CG® from Degussa, common horsechestnut extracts, mallow extracts, witch-hazel extracts, sweet almond extracts, marshmallow root extracts and linseed extracts, for instance ALMONDERMIN LS 3380® from Cognis, burdock extracts, nettle extracts, birch extracts, horsetail extracts, camomile extracts, for instance those sold under the name EXTRAPONE 9 SPECIAL® by Symrise, skullcap extracts, European meadowsweet extracts (for example CYTOBIOL ULMAIRE from Libiol), a mixture of extracts of white ginger, of horsetail, of nettle, of rosemary and of yucca, for instance HERB EXTRACT B1348® from Bell Flavors & Fragrances, extracts of acacia, of elm, of white willow, of cinnamon, of birch and of meadowsweet, Panama sapogenins, zinc phenolsulphonate from Interchemical, extracts of gentian, of cucumber and of walnut, the mixture of extracts of Ratanhia, of grapefruit, of gumweed and of oak gall, for instance EPILAMI® from Alban Muller.

As preferred astringents according to the invention, use will be made of skullcap extracts, European meadowsweet extracts, meadowsweet extracts, gentian extracts and burdock extracts, and mixtures thereof.

### 22. Wound-Healing Agents

Examples of wound-healing agents that may especially be mentioned include:

allantoin, urea, certain amino acids, for instance hydroxyproline, arginine, and serine, and also extracts of white lily (for instance PHYTÉLÈNE LYS 37EG 16295 from Indena), a yeast extract, for instance the wound-healing agent LS LO/7225B from Laboratoires Sérobiologiques), tamanu oil, extract of Saccharomyces cerevisiae, for instance BIODYNES® TRF® from Arch Chemical, oat extracts, chitosan and compounds, for instance chitosan glutamate, carrot extracts, artemia extract, for instance GP4G® from Vincience, sodium acexamate, lavandin extracts, propolis extracts, ximeninic acid and salts thereof, rose hip oil, marigold extracts, for instance SOUCI AMI® LIPOSOLIBLE from Alban Muller, horsetail extracts, lemon peel extracts, for instance HERBASOL® CITRON from Cosmetochem, helichrysum extracts, common yarrow extracts and folic acid.

As preferred wound-healing agents according to the invention, use will be made of arginine, serine, folic acid, tamanu oil, sodium acexamate, horsetail extracts and helichrysum extracts, and mixtures thereof.

### 23. Anti-Inflammatory Agents

As particular anti-inflammatory agents that may be used according to the invention, mention may be made of cortisone, hydrocortisone, indomethacin, betamethasone, azelaic acid, acetaminophen, diclofenac, clobetasol propionate, folic acid; an extract of Eperua falcata bark, such as the product sold by Cognis under the name EPERULINE®; an extract of Paeonia suffruticosa root, such as the product sold by Ichimaru Pharcos under the name BOTANPI LIQUID B®; and mixtures thereof.

Preferred anti-inflammatory agents that will be mentioned are azelaic acid, folic acid, an extract of Eperua falcata bark, such as the product sold by Cognis under the name EPERULINE®; an extract of Paeonia suffruticosa root, such as the product sold by Ichimaru Pharcos under the name BOTANPI LIQUID B®; and mixtures thereof.

### 24. Anti-Acne Agents

In one advantageous aspect of the invention, the composition may also comprise at least one anti-acne active agent.

The term “anti-acne active agent” especially means any active agent that has effects on the specific flora of greasy skin, for instance Propionibacterium acnes (P. acnes).

These effects may be bactericidal.

Antibactericidal active agents that may especially be mentioned include:
active agents and preserving agents with antimicrobial activity mentioned in Patent Application DE 103 24 567, which is incorporated into the present invention by reference;
Asiatic acid;
the monoethanolamine salt of 1-hydroxy-4-methyl-6-trimethylpentyl-2-pyridone (INCI name: piroctone olamine), sold especially under the name OCTOPIROX® by Clariant;
citronellic acid, perillic acid (or 4-iso-propenylcyclohex-1-enecarboxylic acid);
2-ethylhexyl glyceryl ether (INCI name: ethylhexylglycerine), for example sold under the name SENSIVA SC 50® by Shulke & Mayr;
glyceryl caprylate/caprate, for example sold under the name CAPMUL MCM® by Abitec;
sodium calcium phosphosilicate, especially sold under the names BIOACTIVE GLASSPOWDER® and ACTYSSE PREMIER BG® by Schott Glass;
silver-based particles, for example those sold under the name METASHINE ME 2025 PS® by Nippon Sheet Glass;
hop cone extract (Humulus lupulus) obtained by supercritical CO₂ extraction, such as that sold under the name HOP CO2-TO EXTRACT® by Flavex Naturextrakte;
St. John's Wort extract obtained by supercritical CO₂ extraction, such as that sold under the name ST. JOHN'S WORT CO2-TO EXTRACT® by Flavex Naturextrakte;
the mixture of extracts of roots of Scutellaria baicalensis, of Paeonia suffruticosa and Glycyrrhiza glabra, such as that sold under the name BMB-CF® by Naturogin;
argan tree extract, for instance ARGAPURE LS9710® from Cognis;
bearberry leaf extracts, for instance that sold under the name MELFADE-J by Pentapharm;
10-hydroxy-2-decanoic acid such as ACNACIDOL P® from Vincience, sodium ursolate, azelaic acid, diiodomethyl p-tolyl sulphone such as AMICAL FLOWABLE® from Angus, malachite powder, zinc oxide such as ZINCARE® from Elementis GmbH, octadecenedioic acid such as ARLATONE DIOIC DCA from Uniqema; ellagic acid; 2,4,4′-trichloro-2′-hydroxydiphenyl ether (or triclosan), 1-(3′,4′-dichlorophenyl)-3-(4′-chloro-phenyl)urea (or triclocarban), 3,4,4′-trichloro-carbanilide, 3′,4′,5′-trichlorosalicylanilide, phenoxy-ethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, 3,4,4′-trichloro-carbanalide, octoxyglycerine or octoglycerine, octanoylglycine such as LIPACID C8G® from SEPPIC, caprylyl glycol, 10-hydroxy-2-decanoic acid, dichlorophenylimidazoldioxolane and compounds thereof described in Patent Application WO 93/18743, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts; and
mixtures thereof.

Mention may also be made of certain surfactants with an antimicrobial effect, for instance sodium cocoamphoacetate or disodium diacetate such as MIRANOL C2M CONC. NP, betaines, for instance the cocoyl betaine GENAGEN KB from Clariant, sodium lauryl ether sulphate, for instance EMAL 270 D from Kao, decyl glucoside, for instance PLANTACARE 2000 UP, branched C₁₂₋₁₃ dialkyl malates, for instance COSMACOL EMI, propylene glycol monoesters, for instance propylene glycol monolaurate, monocaprylate or monocaprate, lauryldimethylamine betaine, for instance EMPIGEN BB/LS, and also polyquaternary ammoniums such as QUATERNIUM-24 or BARDAC 2050 from Lonza and those described in Patent FR 0 108 283, and mixtures thereof.

As preferred antimicrobial agents, an agent chosen from octoglycerine or octoxyglycerine, and 10-hydroxy-2-decanoic acid, and mixtures thereof, will be used in the compositions of the invention.

Other additional anti-acne active agents may be added to the abovementioned anti-acne active agents.

Mention may be made especially of active agents with bacterial anti-adhesion effects or agents that act on the biofilm of bacteria to prevent them from multiplying.

As agents for preventing and/or reducing the adhesion of microorganisms, mention may be made especially of: phytanetriol and compounds thereof as described in Patent Application EP 1 529 523, plant oils such as wheatgerm oil, calendula oil, castor oil, olive oil, avocado oil, sweet almond oil, groundnut oil, jojoba oil, sesame seed oil, apricot kernel oil, sunflower oil and macadamia oil, described in Patent EP 1 133 979, or certain surfactants such as disodium cocoamphodiacetate, oxyethylenated (7 EO) glyceryl cocoate, 18-hexadecenylsuccinate, octoxyglyceryl palmitate, octoxyglyceryl behenate, dioctyl adipate, PPG-15 stearyl ether, and the branched C₁₂-C₁₃ dialcohol tartrates described in Patent EP 1 129 694, and mixtures thereof.

In particular with regard to the propagation of P. acnes, or as active agents that act on the biofilm of bacteria to prevent them from proliferating, mention may be made of pentylene glycol, Nylon-66 (polyamide 66 fibres), rice bran oil, polyvinyl alcohol such as CELVOL 540 PV ALCOHOL® from Celanese Chemical, rapeseed oil such as AKOREX L® from Karlshamns, and fructose compounds, and mixtures thereof.

The anti-acne active agent may be present in a content ranging from 0.01% to 10% by weight and preferably from 0.05% to 5% by weight relative to the total weight of the composition.

As a function of the nature and/or solubility of the abovementioned active agents, a person skilled in the art will know how to select the most suitable embodiment according to the invention.

As lipophilic active agents that may be used in the kit or at least one of the compositions of the invention, mention may be made especially of D-α-tocopherol, DL-α-tocopherol, D-α-tocopheryl acetate, DL-α-tocopheryl acetate, ascorbyl palmitate, vitamin F glycerides, D vitamins, vitamin D2, vitamin D3, retinol, retinol esters, retinyl palmitate, retinyl propionate, carotenes including β-carotene, D-panthenol, farnesol, farnesyl acetate, salicylic acid and compounds thereof, for instance 5-n-octanoylsalicylic acid, alkyl esters of α-hydroxy acids such as citric acid, lactic acid, glycolic acid, asiatic acid, madecassic acid, asiaticoside, the total extract of Centella asiatica, β-glycyrrhetinic acid, α-bisabolol, ceramides, for instance 2-oleoylamino-1,3-octadecane, phytanetriol, phospholipids of marine origin rich in polyunsaturated essential fatty acids, ethoxyquine, rosemary extract, balm extract, quercetin, extract of dried microalgae, essential oil of bergamot, octyl methoxycinnamate, butylmethoxydibenzoylmethane, octyl triazone, 3,5-di-tert-butyl-4-hydroxy-3-benzylidenecamphor, antibiotics, antifungal agents, anaesthetics, analgesics, antiseptics, antiviral agents, pesticides and herbicides, and mixtures thereof.

The cosmetic and/or dermatological active agents will be present in the kit or one of the compositions according to the invention in a content ranging from 0.001% to 20% by weight relative to the total weight of the composition, preferably from 0.01% to 10%, even more preferentially from 0.5% to 5% and more preferably still from 0.1% to 1% by weight relative to the total weight of the composition.

For “peeling” applications, the contents of cosmetic and/or dermatological active agents may range from 1% to 50% by weight relative to the total weight of the composition and preferably from 1% to 30% by weight relative to the total weight of the composition.

Peeling is a well-known means for improving the appearance and/or texture of the skin and/or the scalp, especially for improving the radiance and homogeneity of the complexion and/or for reducing the visible and/or tactile irregularities of the skin, and in particular for improving the surface appearance of the skin, for attenuating actinic lentigo, acne or chicken pox scars, and also for preventing, attenuating or combating the signs of ageing of the skin, and especially for smoothing out irregularities in the texture of the skin, such as wrinkles and fine lines.

It has the effect of removing a surface part of the skin to be treated (epidermis and possibly the upper layer of the dermis), via chemical methods.

Other Additional Ingredients

To complement and/or optimize the effects imparted by the cosmetic and/or dermatological active agents mentioned above on the keratinous substances, it may be advantageous to incorporate into the compositions of the invention other additional ingredients.

In particular, these additional ingredients may impart an immediate visual effect that will be relayed by the biological effect of the active agents mentioned above.

They may also, via a mechanical action (e.g.: abrasive fillers), amplify the effect of the biological active agents mentioned above.

Thus, the composition according to the invention may also comprise at least one agent chosen from matifying agents, fillers with a soft-focus effect, fluorescent agents, agents for favouring the naturally pink coloration of the skin, abrasive fillers or exfoliants, and mixtures thereof.

### Matifying Agents

The term “matifying agent” is understood to mean agents intended to make the skin visibly more matt, less shiny.

The matifying effect of the agent and/or of the composition containing it may especially be evaluated using a gonioreflectometer, by measuring the ratio R between the specular reflection and the diffuse reflection. An R value less than or equal to 2 generally indicates a matifying effect.

The matifying agent may especially be chosen from a rice starch, a maize starch, kaolinite, silicas, talc, pumpkin seed extract, cellulose microbeads, plant fibres, synthetic fibres, in particular polyamide fibres, expanded acrylic copolymer microspheres, polyamide powders, silica powders, polytetra-fluoroethylene powders, silicone resin powders, acrylic polymer powders, wax powders, polyethylene powders, crosslinked organopolysiloxane elastomer powders coated with silicone resin, composite talc/titanium dioxide/alumina/silica powders, amorphous mixed silicate powders, silicate particles and especially mixed silicate particles, and mixtures thereof.

As examples of matifying agents, mention may especially be made of:
rice or maize starch, in particular an aluminium starch octenylsuccinate sold under the name DRY-FLO® by National Starch;
kaolinite;
silicas;
talc;
a pumpkin seed extract as sold under the name CURBILENE® by Indena;
cellulose microbeads as described in Patent Application EP 1 562 562;
fibres, such as silk fibres, cotton fibres, wool fibres, flax fibres, fibres of cellulose extracted especially from wood, vegetables or algae, polyamide (Nylon®) fibres, modified cellulose fibres, poly(p-phenylene terephthalamide) fibres, acrylic fibres, polyolefin fibres, glass fibres, silica fibres, aramid fibres, carbon fibres, Teflon® fibres, insoluble collagen fibres, polyester fibres, polyvinyl chloride or polyvinylidene chloride fibres, polyvinyl alcohol fibres, polyacrylonitrile fibres, chitosan fibres, polyurethane fibres, polyethylene phthalate fibres, fibres formed from a blend of polymers, resorbable synthetic fibres, and mixtures thereof described in Patent Application EP 1 151 742;
expanded acrylic copolymer microspheres such as those sold by Expancel under the name EXPANCEL 551®;
fillers having an optical effect as described in Patent Application FR 2 869 796, in particular:
   polyamide (Nylon®) powders, such as for example ORGASOL-type nylon-12 particles from Arkema, having an average size of 10 microns and a refractive index of 1.54;
   silica powders, such as for example the silica beads SB150 from Miyoshi, having an average size of 5 microns and a refractive index of 1.45;
   polytetrafluoroethylene powders, such as the CERIDUST 9205F PTFEs from Clariant, having an average size of 8 microns and a refractive index of 1.36;
   silicone resin powders such as the TOSPEARL 145A silicone resin from GE Silicone, having an average size of 4.5 microns and a refractive index of 1.41;
   acrylic copolymer powders, especially polymethyl (meth)acrylate powders, such as the JURYMER MBI PMMA particles from Nihon Junyoki, having an average size of 8 microns and a refractive index of 1.49, or the MICROPEARL M100® and F 80 ED® particles from Matsumoto Yushi-Seiyaku;
   wax powders such as MICROEASE 114S paraffin wax particles from MicroPowders, having an average size of 7 microns and a refractive index of 1.54;
   polyethylene powders, especially comprising at least one ethylene/acrylic acid copolymer, and in particular formed from ethylene/acrylic acid copolymers such as FLOBEADS EA 209 particles from Sumitomo (having an average size of 10 microns and a refractive index of 1.48);
   crosslinked organopolysiloxane elastomer powders coated in silicone resin, especially silsesquioxane resin, as described, for example, in U.S. Pat. No. 5,538,793. Such elastomer powders are sold under the names KSP-100, KSP-101, KSP-102, KSP-103, KSP-104 and KSP-105 by Shin-Etsu; and
   composite talc/titanium dioxide/alumina/silica powders such as those sold under the name COVERLEAF® AR-80 by Catalyst & Chemicals, and mixtures thereof,
   compounds that absorb and/or adsorb sebum as described in Patent Application FR 2 869 796. Mention may especially be made of:
   silica powders, such as for example porous silica microspheres sold under the name SILICA BEADS SB-700 sold by Myoshi, SUNSPHERE® H51, SUNSPHERE® H33 and SUNSPHERE® H53 sold by Asahi Glass; amorphous silica microspheres coated with polydimethylsiloxane sold under the name SA SUNSPHERE® H-33 and SA SUNSPHERE® H53 sold by Asahi Glass;
   amorphous mixed silicate powders, especially of aluminium and magnesium, such as for example the one sold under the name NEUSILIN UFL2 by Sumitomo;
   polyamide (Nylon®) powders, such as for example ORGASOL® 4000 sold by Arkema, and;
   acrylic polymer powders, especially polymethyl methacrylate powders, such as for example COVABEAD® LH85 sold by Wackherr; polymethyl methacrylate/ethylene glycol dimethacrylate powders, such as for example DOW CORNING 5640 MICROSPONGE® SKIN OIL ADSORBER sold by Dow Corning, or GANZPEARL® GMP-0820 sold by Ganz Chemical; polyallyl methacrylate/ethylene glycol dimethacrylate powders, such as for example POLY-PORE® L200 or POLY-PORE® E200 sold by AMCOL; ethylene glycol dimethacrylate/lauryl methacrylate copolymer powders, such as for example POLYTRAP® 6603 sold by Dow Corning;
   silicate particles, such as alumina silicate; and
   mixed silicate particles, such as:
      magnesium-aluminium silicate particles, such as saponite or magnesium-aluminium silicate hydrated with a sodium sulphate sold under the trade name SUMECTON® by Kunimine; and
      the complex of magnesium silicate, hydroxyethyl cellulose, black cumin oil, pumpkin oil and phospholipids, or MATIPURE® from Lucas Meyer, and
   mixtures thereof.

As preferred matifying agents, it will be possible to use, according to the invention, a pumpkin seed extract, a rice or maize starch, kaolinite, silicas, talc, polyamide powders, polyethylene powders, acrylic copolymer powders, expanded acrylic copolymer microspheres, silicon resin microbeads, mixed silicate particles and mixtures thereof.

Fillers with a Soft-Focus Effect

These fillers may be any material capable of modifying and hiding wrinkles by virtue of their intrinsic physical properties. These fillers may especially modify wrinkles via a tightening effect, a covering effect or a soft-focus effect.

Examples of fillers that may be given include the following compounds:
porous silica microparticles, for instance the SILICA BEADS SB150 and SB700 from Miyoshi with a mean size of 5 μm; the series-H SUNSPHERES from Asahi Glass, for instance SUNSPHERES H33, H51 with respective sizes of 3.5 and 5 μm;
hollow hemispherical silicone resin particles such as NLK 500®, NLK 506® and NLK 510° from Takemoto Oil and Fat, especially described in EP-A-1 579 849;
silicone resin powders, for instance the silicone resin TOSPEARL® 145A from GE Silicones, with a mean size of 4.5 μm;
acrylic copolymer powders, especially polymethyl (meth)acrylate powders, for instance the PMMA particles JURYMER MBI® from Nihon Junyoki, with a mean size of 8 μm, the hollow PMMA spheres sold under the name COVABEAD® LH85 by the company Wackherr, and vinylidene/acrylonitrile/methylene methacrylate expanded microspheres sold under the name EXPANCEL®;
wax powders, for instance the paraffin wax particles MICROEASE® 114S from MicroPowders, with a mean size of 7 μm;
polyethylene powders, especially comprising at least one ethylene/acrylic acid copolymer, for instance the FLOBEADS® EA 209 particles from Sumitomo, with a mean size of 10 μm;
crosslinked elastomeric organopolysiloxane powders coated with silicone resin and especially with silsesquioxane resin, under the names KSP-100®, KSP-101®, KSP-102®, KSP-103®, KSP-104® and KSP-105® by Shin-Etsu;
talc/titanium dioxide/alumina/silica composite powders, for instance those sold under the name COVERLEAF AR-80® by Catalyst & Chemicals;
talc, mica, kaolin, lauryl glycine, starch powders crosslinked with octenyl succinate anhydride, boron nitride, polytetrafluoroethylene powders, precipitated calcium carbonate, magnesium carbonate, magnesium hydrogen carbonate, barium sulphate, hydroxyapatite, calcium silicate, cerium dioxide and glass or ceramic microcapsules;
hydrophilic or hydrophobic, synthetic or unnatural, mineral or organic fibres such as silk fibres, cotton fibres, wool fibres, flax fibres, cellulose fibres extracted especially from wood, vegetables or algae, polyamide (Nylon®) fibres, modified cellulose fibres, poly-p-terephthamide fibres, acrylic fibres, polyolefin fibres, glass fibres, silica fibres, aramid fibres, carbon fibres, polytetrafluoroethylene (Teflon®) fibres, insoluble collagen fibres, polyester fibres, polyvinyl chloride fibres, polyvinylidene chloride fibres, polyvinyl alcohol fibres, polyacrylonitrile fibres, chitosan fibres, polyurethane fibres, polyethylene phthalate fibres, fibres formed from a blend of polymers, resorbable synthetic fibres, and mixtures thereof described in Patent Application EP 1 151 742;
spherical elastomeric crosslinked silicones, for instance TREFIL E-505C® or E-506C® from Dow Corning;
abrasive fillers, which, via a mechanical effect, smooth out the skin microrelief, such as abrasive silica, for instance ABRASIF SP® from Semanez or nut or shell powders (for example of apricot or walnut, from Cosmétochem).

The fillers with an effect on the signs of ageing are especially chosen from porous silica microparticles, hollow hemispherical silicone particles, silicone resin powders, acrylic copolymer powders, polyethylene powders, crosslinked elastomeric organopolysiloxane powders coated with silicone resin, talc/titanium dioxide/alumina/silica composite powders, precipitated calcium carbonate, magnesium carbonate, magnesium hydrogen carbonate, barium sulphate, hydroxyapatite, calcium silicate, cerium dioxide, glass or ceramic microcapsules, and silk fibres or cotton fibres, and mixtures thereof.

The term “soft-focus filler” is understood to mean a filler which also gives transparency to the skin tone and a blurred effect. Preferably, the soft-focus fillers have an average particle size less than or equal to 15 microns. These particles may be of any shape and in particular may be spherical or non-spherical. More preferably, these fillers are non-spherical.

The soft-focus fillers may be chosen from silica and silicate powders, especially alumina powders, polymethyl methacrylate (PMMA) type powders, talc, silica/TiO₂ or silica/zinc oxide composites, polyethylene powders, starch powders, polyamide powders, styrene/acrylic copolymer powders, silicone elastomers, and mixtures thereof.

In particular, mention may be made of talc having a number-average size less than or equal to 3 microns, for example talc having a number-average size of 1.8 microns and especially that sold under the trade name TALC P3® from Nippon Talc, nylon-12 powder, especially that sold under the name ORGASOL 2002 EXTRA D NAT COS® from Atochem, silica particles that are surface-treated with a 1 to 20 mineral wax (INCI name: hydrated silica (and) paraffin) such as those sold by Degussa, amorphous silica microspheres, such as those sold under the name SUNSPHERE for example having the reference H-53® by Asahi Glass, and silica microbeads such as those sold under the name SB-700® or SB-150® by Miyoshi, this list not being limiting.

The concentration of these fillers having an effect on the signs of ageing in the compositions according to the invention may be between 0.1 and 400 or even between 0.1 and 20% by weight relative to the total weight of the composition.

### Fluorescent Agents

The term “fluorescent agent” is understood to mean a substance which, under the effect of ultraviolet rays and/or visible light, re-emits into the visible range the portion of light that it has absorbed in the same colour that it reflects naturally. The naturally reflected colour is thus reinforced by the re-emitted colour and appears extremely bright.

Mention may be made, for example, of coloured resins of polyamide and/or of formaldehyde/benzoguanamine and/or of melamine/formaldehyde/sulphonamide, among the coloured aminotriazine/formaldehyde/sulphonamide co-condensates and/or among the metallized polyester flakes and/or mixtures thereof. These fluorescent pigments may also be in the form of aqueous dispersions of fluorescent pigments.

Mention may also be made of the pink-coloured fluorescent aminotriazine/formaldehyde/sulphonamide co-condensate having an average particle size of 3-4 microns, sold under the trade name FIESTA ASTRAL PINK FEX-1 and the blue-coloured fluorescent aminotriazine/formaldehyde/sulphonamide co-condensate having an average particle size of 3-4.5 microns sold under the trade name FIESTA COMET BLUE FTX-60 by Swada or else the benzoguanamine/formaldehyde resin coated with formaldehyde/urea resin and coloured yellow, sold under the trade name FB-205 YELLOW and the benzoguanamine/formaldehyde resin coated with formaldehyde/urea resin and coloured red, sold under the trade name FB-400 ORANGE RED by UK Seung Chemical, the orange-coloured polyamide resin sold under the trade name FLARE 911 ORANGE 4 by Sterling Industrial Colors.

The fluorescent substances are preferably present in the composition in an amount ranging from 0.1 to 20%, preferably from 0.1 to 15%, more preferably from 0.5 to 3% by weight, relative to the total weight of the composition.

When the organic fluorescent substances are white, they are also called optical brighteners.

The optical brightener has the effect of intensifying the radiance and brightening the tones of the cosmetic compositions containing them upon application to the skin.

Among the optical brighteners, mention may more particularly be made of stilbene compounds, in particular polystyrylstilbenes and triazinestilbenes, coumarin compounds, in particular hydroxycoumarins and aminocoumarins, oxazole compounds, benzoxazole, imidazole, triazole, pyrazoline, pyrene compounds and porphyrin compounds and/or mixtures thereof.

Such compounds are, for example, available under the trade names TINOPAL SOP® and UVITEX OB® from Ciba Geigy.

The optical brighteners preferentially used are sodium 4,4′-bis(4,6-dianilino-1,3,5-triazin-2-yl)amino]-stilbene-2,2′-disulphonate, 2,5-thiophendiylbis(5-tert-butyl-1,3-benzoxazole), disodium distyryl-4,4′-biphenyl sulphonate and/or mixtures thereof.

### Agents Favouring the Naturally Pink Coloration of the Skin

Mention may especially be made of:
a self-tanning agent, that is to say an agent which, when applied to the skin, especially to the face, gives a tanning effect that is more or less similar in appearance to that which may result from prolonged exposure to sunlight (natural tan) or under a UV lamp;
an additional colouring agent, that is to say any compound having a particular affinity for the skin that allows it to give the skin a lasting, non-covering (i.e. that does not have a tendency to opacify the skin) coloration and that is not removed either with water or using a solvent, and that withstands both rubbing and washing with a solution containing surfactants. Such a lasting coloration is therefore distinguished from the superficial and transient coloration provided, for example, by a makeup pigment; and mixtures thereof.

As examples of self-tanning agents, mention may be made of:
dihydroxyacetone (DHA);
erythrulose; and
the combination of a catalytic system formed from:
   manganese and/or zinc salts and oxides; and
   alkali-metal and/or alkaline-earth-metal hydrogencarbonates.

The self-tanning agents are generally chosen from moncarbonylated or polycarbonylated compounds such as, for example, isatin, alloxan, ninhydrin, glyceraldehydes, mesotartric aldehyde, glutaraldehyde, erythrulose, pyrazolin-4,5-dione compounds as described in Patent Application FR 2 466 492 and WO 97/35842, dihydroxyacetone (DHA), 4,4-dihydroxypyrazolin-5-one compounds as described in Patent Application EP 903 342. Preferably, DHA will be used.

DHA may be used in free and/or encapsulated form, for example in lipid vesicles such as liposomes, especially described in Application WO 97/25970.

Generally, the self-tanning agent is present in an amount ranging from 0.01 to 20 wt %, and preferably in an amount between 0.1 and 10 wt % of the total weight of the composition.

As additional colouring agents, mention may be made, for example, of plant extracts such as, for example, the sorghum extracts obtained from the whole plant, the stems, the seeds or the leaves of the genus Sorghum. The preferred species of Sorghum are chosen from Sorghum bicolor, Sorghum caudatum, Sorghum nervosum, Sorghum durra, Sorghum vulgare and Sorghum plants in combination with Colletotrichum graminicola such as those described in Patent Application FR 0 200 251.

It is also possible to use other colorants that enable the colour produced by the self-tanning agent to be modified.

These colorants may be chosen from direct synthetic or natural colorants.

These colorants may be chosen, for example, from red or orange colorants of the fluorane type such as those described in Patent Application FR 2 840 806. Mention may be made, for example, of the following colorants:
tetrabromofluorescein or eosin known under the CTFA name: CI-45380 or Red 21;
phloxine B known under the CTFA name: CI-45410 or Red 27;
diiodofluorescein known under the CFTA name: CI-45425 or Orange 10;
dibromofluorescein known under the CTFA name: CI-45370 or Orange 5;
the sodium salt of tetrabromofluorescein known under the CTFA name: CI-45380 (Na salt) or Red 22;
the sodium salt of phloxine B known under the CTFA name: CI-45410 (Na salt) or Red 28;
the sodium salt of diiodofluorescein known under the CTFA name: CI-45425 (Na salt) or Orange 11;
erythrosine known under the CTFA name: CI-45430 or Acid Red 51; and
phloxine known under the CTFA name: CI-45405 or Acid Red 98.

These colorants may also be chosen from anthraquinones, caramel, carmine, carbon black, azulene blues, methoxalene, trioxalene, guajazulene, chamuzulene, rose bengal, cosine 10B, cyanosine, and daphnin.

These colorants may also be chosen from indole compounds such as monohydroxyindoles as described in Patent FR 2 651 126 (i.e. 4-, 5-, 6- or 7-hydroxy-indole) or dihydroxyindoles as described in Patent EP-B-0 425 324 (i.e. 5,6-dihydroxyindole, 2-methyl-5,6-dihydroxyindole, 3-methyl-5,6-dihydroxyindole, 2,3-dimethyl-5,6-dihydroxyindole).

### Abrasive Fillers or Exfoliating Agents

As exfoliating agents that can be used in the rinse-off compositions according to the invention, mention may be made, for example, of exfoliating or scrubbing particles of mineral, plant or organic origin. Thus, it is possible to use, for example, polyethylene beads or powder, nylon powder, polyvinyl chloride powder, pumice, ground products of apricot kernels or of nut shells, sawdust, glass beads, alumina, and mixtures thereof.

Mention may also be made of EXFOGREEN® from Solabia (bamboo extract), extracts of strawberry achenes (strawberry achenes from Greentech), peach kernel powder, apricot kernel powder, and finally in the field of plant powders having an abrasive effect, mention is made of cranberry seed powder.

As abrasive fillers or exfoliating agents that are preferred according to the invention, mention will be made of peach kernel powder, apricot kernel powder, cranberry seed powder, extracts of strawberry achenes and bamboo extracts.

The additional ingredient or ingredients used in the kit or one of the compositions according to the invention may represent from 0.0001 to 20%, preferably from 0.01 to 10% and better still from 0.01 to 1% by weight relative to the total weight of the composition.

According to one particular embodiment, the composition according to the invention contains at least urea or one of its compounds, such as HYDROVANCE® from National Starch.

According to another embodiment, the composition according to the invention contains at least acrylic acid homopolymers such as LIPIDURE-HM® from NOF Corporation.

According to another embodiment, the composition according to the invention contains at least spheres of collagen and of chondroitin sulphate of marine origin (Atelocollagen), such as those sold by Engelhard Lyon under the name marine filling spheres.

In particular, said combination between the C-glycoside compound and the spheres of collagen and of chondroitin sulphate of marine origin and/or said composition containing it is used according to the invention to improve the hydration of the skin.

According to another embodiment, the composition according to the invention contains at least hyaluronic acid or spheres of hyaluronic acid.

In particular, said combination between the C-glycoside compound and hyaluronic acid or hyaluronic acid spheres and/or said composition containing it is used according to the invention to improve the hydration of the skin.

According to another embodiment, the composition according to the invention contains at least kojic acid.

In particular, said combination between the C-glycoside compound and kojic acid and/or said composition containing it is used according to the invention to improve the depigmentation of the skin.

According to another embodiment, the composition according to the invention contains at least ellagic acid.

In particular, said combination between the C-glycoside compound and ellagic acid and/or said composition containing it is used according to the invention to improve the depigmentation of the skin.

According to another embodiment, the composition according to the invention contains at least n-octanoyl-5-salicylic acid also known by the INCI name capryloyl salicylic acid.

According to another embodiment, the composition according to the invention contains at least 8-hexa-decene-1,16-dicarboxylic acid or 9-octadecenedioic acid and compounds thereof.

According to another embodiment, the composition according to the invention contains at least 4-(2-hydroxyethyl)piperazine-1-propanesulphonic acid (HEPES).

According to another embodiment, the composition according to the invention contains at least 2-oxo-thiazolidine-4-carboxylic acid (procysteine) and compounds thereof.

According to another embodiment, the composition according to the invention contains at least {2-[acetyl-(3-trifluoromethylphenyl)amino]-3-methyl-butyrylamino}acetic acid, otherwise known as N—[N-acetyl-N′-(3-trifluoromethyl)phenylvalyl]glycine or N-acetyl-N-[3-(trifluoromethyl)phenyl]valylglycine or acetyl trifluoromethyl phenyl valylglycine.

According to another embodiment, the composition according to the invention contains at least one sphingosine such as the salicyloyl sphingosine sold under the name “PHYTOSPHINGOSINE® SLC by Degussa.

In particular, the combination between a C-glycoside compound and at least one phytosphingosine and/or said composition containing it is used according to the invention to improve the biomechanical properties of the skin.

According to another embodiment, the composition according to the invention contains at least one ceramide or compound, in particular ceramides of type 2 (such as N-oleoyldihydrosphingosine), of type 3 (such as stearoyl-4-hydroxysphinganine (INCI name)) and of type 5 (such as N-2-hydroxypalmitoyldihydrosphingosine, having the INCI name: hydroxypalmitoyl sphinganine).

According to another embodiment, the composition according to the invention contains at least one ascorbyl glucoside (vitamin CG).

According to another embodiment, the composition according to the invention contains at least vitamin B3.

According to another embodiment, the composition according to the invention contains at least biotin.

According to another embodiment, the composition according to the invention contains at least caprylyl glycol. In particular, the combination between a C-glycoside compound and at least caprylyl glycol and/or said composition containing it is used according to the invention to improve the hydration of the skin.

According to another embodiment, the composition according to the invention contains at least one phyto-complex comprising cinnamic acid, phloroglucinol and a soybean extract. As the soybean extract, use will be made, in particular, of a hydrolysed soybean protein such as that sold by Silab under the name RIDULISSE®.

In particular, said combination between the C-glycoside compound and the phytocomplex and/or said composition containing it is used according to the invention to prevent and/or treat the hormonal ageing of the area around the lips, in particular of the top lip area, and especially in menopausal women.

According to another embodiment, the composition according to the invention contains at least TOTAROL™ or an extract of Podocarpus totara containing totarol (totara-8,11,13-trienol or 4b,5,6,7,8,8a,9,10-octa-hydro-4b,8,8-trimethyl-1-(1-methylethyl)-2-phenanthrenol.

According to another embodiment, the composition according to the invention contains at least phloretin.

In particular, the combination between a C-glycoside compound and phloretin and/or said composition containing it is used according to the invention to improve the antioxidant effect of the composition.

According to another embodiment, the composition according to the invention contains at least one pomegranate extract.

In particular, the combination between a C-glycoside compound and a pomegranate extract and/or said composition containing it is used according to the invention to improve the antioxidant effect of the composition.

According to another embodiment, the composition according to the invention contains at least ferulic acid.

In particular, the combination between a C-glycoside compound and ferulic acid and/or said composition containing it is used according to the invention to improve the antioxidant effect of the composition.

According to another embodiment, the composition according to the invention contains at least adenosine.

In particular, the combination between a C-glycoside compound and adenosine and/or said composition containing it is used according to the invention to improve the dermo-relaxation and/or dermo-contraction of the skin, and to thus prevent and/or treat, in particular, the expression lines on the face.

According to another embodiment, the composition according to the invention contains at least ginger.

In particular, the combination between a C-glycoside compound and ginger and/or said composition containing it is used according to the invention to improve the dermo-relaxation and/or dermo-contraction of the skin, and to thus prevent and/or treat, in particular, the expression lines on the face.

According to another embodiment, the composition according to the invention contains at least manganese gluconate.

According to another embodiment, the composition according to the invention contains at least one sapogenin or a natural extract containing it, in particular an extract of wild yam.

According to another embodiment, the composition according to the invention contains at least one extract of sea fennel.

In particular, said combination between the C-glycoside compound and the sea fennel extract and/or the composition containing it is used according to the invention to improve collagen synthesis, and thus to improve the biomechanical properties of the skin, in particular the firmness.

According to another embodiment, the composition according to the invention contains at least retinol or one of its compounds, in particular retinyl palmitate.

In particular, said combination between the C-glycoside compound and retinol or one of its compounds and/or the composition containing it is used according to the invention to improve collagen synthesis, and thus to improve the biomechanical properties of the skin, in particular the firmness.

According to another embodiment, the composition according to the invention contains at least one blueberry extract (Vaccinium angustifolium, Vaccinium myrtillus).

In particular, said combination between the C-glycoside compound and the blueberry extract and/or the composition containing it is used according to the invention to favour the synthesis of collagen and/or to prevent its degradation, and/or to favour the stimulation and/or production of glycosaminoglycans.

According to another embodiment, the composition according to the invention contains at least one extract of black tea such as KOMBUCHKA.

In particular, said combination between the C-glycoside compound and the black tea extract and/or the composition containing it is used according to the invention to favour skin microcirculation, and thus, in particular, to improve the appearance of the area around the eye, in particular to reduce dark circles.

According to another embodiment, the composition according to the invention contains at least one extract of black tea combined with at least one extract of brown sugar, a lychee extract or a mixture thereof.

In particular, said combination between the C-glycoside compound and these 3 extracts and/or said composition containing it is used according to the invention to improve the appearance and/or visibility of the pores, especially by reducing the size and/or number of pores visible on the skin of the face, and in particular in the T zone (forehead, nose, cheeks, chin), in particular on the nose and cheeks.

According to another embodiment, the composition according to the invention contains at least methyl-silanol mannuronate.

According to another embodiment, the composition according to the invention contains at least folic acid or vitamin B9.

According to another embodiment, the composition according to the invention contains at least lycopene.

According to another embodiment, the composition according to the invention contains at least one yeast extract, in particular an extract of Saccharomyces cerevisiae.

According to another embodiment, the composition according to the invention contains at least one lupin extract sold by Silab under the trade name STRUCTURINE®.

According to another embodiment, the composition according to the invention contains at least one extract of hydrolysed soybean proteins.

According to another embodiment, the composition according to the invention contains at least rice proteins or peptides.

According to another embodiment, the composition according to the invention contains at least arginine and/or serine.

In particular, the combination between a C-glycoside compound and arginine or serine and/or said composition containing it is used according to the invention to improve and/or strengthen the barrier function and/or to improve the wound-healing process.

According to another particular embodiment, the composition according to the invention contains at least unsaturated omega-3 and/or omega-5 oils such as musk rose oil.

In particular, said combination between a C-glycoside compound and unsaturated omega-3 and/or omega-5 oils and/or the composition containing it is used according to the invention to improve the barrier function of the skin.

According to another particular embodiment, the composition according to the invention contains at least one extract of Artemisia abrotanum such as PULPACTYL from Silab.

In particular, said combination between the C-glycoside compound and the extract of Artemisia abrotanum and/or said composition containing it is used according to the invention to improve the lipostructure of the skin, and thus to reduce the age-related hollowing of the face.

According to another embodiment, the composition according to the invention contains at least one silica/alumina composite colloidal particle, for example those sold by Grace under the names LUDOX AM, LUDOX AM-X 6021, LUDOX HSA and LUDOX TMA.

According to another embodiment, the composition according to the invention contains at least one grafted silicone-based polymer such as those especially sold by 3M under the trade names VS 80, VS 70 and LO21.

According to another embodiment, the composition according to the invention contains at least one rye seed extract, such as that sold by Silab under the name COHELISS®.

In particular, said combination between the C-glycoside compound and the rye seed extract and/or said composition containing it is used according to the invention to improve the biomechanical properties of the skin, in particular its firmness and/or its elasticity.

According to another embodiment, the composition according to the invention contains at least caffeine.

According to another embodiment, the composition according to the invention contains at least ginseng.

According to another embodiment, the composition according to the invention contains at least ginkgo.

According to another embodiment, the composition according to the invention contains at least ruscus.

According to another embodiment, the composition according to the invention contains at least escin.

According to another embodiment, the composition according to the invention contains at least one extract of mint, in particular of mint leaves such as CALMISKIN® from Silab.

In particular, said combination between the C-glycoside compound and the mint extract and/or the composition containing it is used according to the invention to soothe the skin.

According to another embodiment, the composition according to the invention contains at least one extract of filamentous bacteria such as Vitreoscilla filiformis such as described in Patent EP 761 204 and sold by Chimex under the name MEXORYL SBG®.

In particular, said combination between the C-glycoside compound and the extract of Vitreoscilla filiformis and/or the composition containing it is used according to the invention to soothe the skin.

According to another embodiment, the composition according to the invention contains at least one extract of rose petals such as ROSE FLOWER HERBASOL® EXTRACT from Cosmethochem.

In particular, said combination between the C-glycoside compound and the rose extract and/or the composition containing it is used according to the invention to soothe the skin.

According to another embodiment, the composition according to the invention contains at least one fermented extract of Alteromonas sold under the name ABYSSINE®.

In particular, said combination between the C-glycoside compound and the fermented extract of Alteromonas and/or the composition containing it is used according to the invention to soothe the skin.

According to another embodiment, the composition according to the invention contains at least one spring water from the Vichy basin, such as the waters originating from the Célestins, Chomel, Grande-Grille, Hôpital, Lucas and Parc sources, and preferably water from the Lucas source.

In particular, said combination between the C-glycoside compound and the spring water from the Vichy basin and/or the composition containing it is used according to the invention to soothe the skin.

According to another embodiment, the composition according to the invention contains at least one linseed extract such as that sold under the name LINUMINE® by Lucas Meyer.

According to another embodiment, the composition according to the invention contains at least one emulsifying silicone elastomer such as those sold under the names KSG-210, KSG-310, KSG-320, KSG-330, KSG-440, KSG-710, KSG-830, KSG-840 by Shin Etsu.

According to another embodiment, the composition according to the invention contains at least one silicone elastomer, such as an organopolysiloxane elastomer, preferably at least partially crosslinked (e.g. KSG).

According to another embodiment, the composition according to the invention contains at least DHA.

### Cosmetic Assembly

According to another aspect, the invention also relates to a cosmetic assembly comprising:
i) a container delimiting at least one compartment, said container being closed by means of a closing member; and
ii) a composition as described previously placed inside said compartment.

The container may be any suitable form. It may especially be in the form of a bottle, tube, pot, jar, case, sachet or carton.

The closing member may be in the form of a removable stopper, a lid, a cap, a tear-off strip, or a capsule, especially of the type comprising a body attached to the container and a cover cap articulated on the body. It may also be in the form of a member for selectively closing the container, especially a pump, a valve or a flap valve.

The container may be combined with an applicator. The applicator may be in the form of a brush, as described, for example, in Patent FR 2 722 380. The applicator may be in the form of a foam or elastomer block, a felt or a spatula. The applicator may be free (powder puff or sponge) or securely fastened to a wand borne by the closing member, as described, for example, in U.S. Pat. No. 5,492,426. The applicator may be securely fastened to the container, as described, for example, in Patent FR 2 761 959.

The product may be contained directly in the container, or indirectly. By way of example, the product may be placed on an impregnated support, especially in the form of a wipe or a pad, and placed (individually or in plurality) in a box or in a sachet. Such a support incorporating the product is described, for example, in Application WO 01/03538.

The closing member may be coupled to the container by screwing. Alternatively, the coupling between the closing member and the container is done other than by screwing, especially via a bayonet mechanism, by snap-fastening, gripping, welding, bonding or by magnetic attraction. The term “snap-fastening” is understood to mean, in particular, any system that involves crossing a band or strip of material via elastic deformation of one portion, especially of the closing member, followed by return to the elastically unconstrained position of said portion after crossing of the band or strip.

The container may be at least partially produced from a thermoplastic. As examples of thermoplastic materials, mention may be made of polypropylene or polyethylene.

Alternatively, the container is produced from a non-thermoplastic material, especially glass or metal (or alloy).

The container may have rigid walls or deformable walls, especially in the form of a tube or a tubular bottle.

The container may comprise means intended for initiating or facilitating the distribution of the composition. By way of example, the container may have deformable walls so as to allow the composition to exit in response to a positive pressure inside the container, this positive pressure being caused by elastic (or non-elastic) squeezing of the walls of the container.

The container may be formed from a carton having a base that delimits at least one housing containing the composition, and a lid, especially articulated onto the base, and capable of at least partially covering said base. Such a carton is described, for example, in Application WO 03/018423 or in Patent FR 2 791 042.

The container may be equipped with a squeezing member placed in the vicinity of the container opening. Such a squeezing member makes it possible to wipe the applicator and optionally the wand to which it may be attached. Such a squeezing member is described, for example, in Patent FR 2 792 618.

The content of the aforementioned patents or patent applications is incorporated by reference into the present application.

According to a particular mode of the invention, the assembly may comprise:
a composition A containing at least one C-glycoside compound and optionally an ingredient favouring its solubilization and/or its stabilization and/or its activity; and
a composition B, packaged separately to the composition A, comprising at least one additional cosmetic or dermatological active ingredient.

In this case, the compositions A and B may be packaged separately inside two compartments, formed either from two separate containers, or inside a single device.

The term “single device” is understood to mean a device for which the two compartments are securely fastened to each other. Such a device may be obtained by a process of one-piece moulding via two compartments, especially from a thermoplastic. It may also result from any form of assembly, especially by bonding, welding or else snap-fastening.

According to a first embodiment, the two containers are independent of one another. Such containers may be in various forms. They may especially be tubes, bottles or cans.

One and/or the other of the containers may be topped with a manually activated pump that is topped with a pushbutton for activating the pump and distributing the composition via at least one distribution orifice.

Alternatively, one and/or the other of the containers is pressurized, especially by means of a propellant, in particular a propellent gas. In this case, the container(s) is (are) equipped with a valve topped by a pushbutton equipped with a nozzle or any other dispersal means for distributing the product.

The propellant may be mixed with the composition to be distributed or may be separate from it, in particular via a piston capable of sliding inside the container, or via the flexible walls of a pouch inside which the composition is placed.

The containers may be formed from various materials: plastic, glass or metal.

Alternatively again, the two compartments are formed from two concentric compartments formed inside a tube, and are topped with an airless pump equipped with a pushbutton having one or two distribution orifices. A piston is provided inside the tube which gradually rises in the direction of the pump as the compositions are removed from inside the containers. Such distribution methods are used especially for distributing toothpastes.

The invention also relates to a cosmetic method for caring for and/or cleansing and/or making up the skin or its appendages that comprises applying a composition as defined previously to the skin or its appendages.

The term “appendages” is understood according to the invention to mean the skin, nails, eyelashes and/or body hair, and head hair.

Preferably, this will be a composition for the skin.

In particular, the method according to the invention is intended to reduce the cutaneous imperfections linked to ageing, in particular to actinic ageing.

The term “cutaneous imperfections linked to skin ageing”, in particular to actinic ageing, is understood to mean, in particular, a loss of firmness and/or elasticity and/or tone and/or suppleness of the skin, the formation of wrinkles and fine lines, expression lines, in particular on the forehead and in the space between the eyebrows, perioral wrinkles and fine lines, and/or slackening in the area around the lips, in particular in the top lip area (area between the top lip and the nose), a dull appearance of the complexion, appearance of darkening and/or yellowing of the skin, and/or appearance of senescence spots or “age spots”, the heterogeneity of the complexion (age spots, actinic lentigo), the appearance and/or visibility of the pores and the papery appearance of the skin.

It will especially be intended for people with mature, even very mature, skin.

The term “mature skin” is understood according to the invention to mean, in particular, people who are at least 40 years old.

The term “very mature skin” is understood according to the invention to mean, in particular, people who are at least 50 years old, in particular at least 60 years old, even 65 years old.

The compositions and/or combinations according to the invention intended to prevent and/or smooth out the expression lines will be applied around the orifices that form the nose (nasal grooves), the mouth (perioral wrinkles and so-called bitterness lines) and the eyes (crow's foot wrinkles), around which lie the skin muscles, and also between the eyebrows (glabella or lion wrinkles) and on the forehead. In particular, it will be sought to prevent and/or smooth out the wrinkles on the forehead and in the area between the eyebrows.

The compositions and/or combinations according to the invention intended to prevent and/or treat the ageing of the area around the lips will, in particular, be applied to menopausal women, in particular in the top lip area.

The compositions and/or combinations according to the invention intended to prevent and/or treat the papery appearance of the skin will, in particular, be applied to the back of the hands.

The compositions and/or combinations according to the invention intended to reduce the appearance and/or visibility of the pores will be applied, in particular, to the T zone (forehead, nose, cheeks, chin) and especially in Asiatic or Caucasian populations.

According to another embodiment, it is intended to reduce the cutaneous imperfections of greasy skin, in particular to matify the skin.

The term “cutaneous imperfections of greasy skin” is understood, in particular according to the invention, to mean aesthetic disorders such as shiny skin, a poorer staying power for makeup, a thick skin grain generally associated with a desquamating defect, pronounced pores, a skin whose follicular orifices are dilated or filled with minute horny spicules, or even with comedones or blackheads (resulting however more from a retention phenomenon that from an increase in excretion).

The term “matify” is understood to mean to make the skin visibly more matt, less shiny. The matifying effect of the composition may especially be evaluated using a gonioreflectometer, by measuring the ratio R between the specular reflection and the diffuse reflection. An R value less than or equal to 2 generally indicates a matifying effect.

In particular, the composition is applied to the areas of the face or the forehead having shiny skin.

According to a particular mode of the invention, the method is intended for the care of people having dark skin (especially skin of phototype IV to VI).

According to another mode of the invention, the method is intended for the care of people having light skin (especially skin of phototype I to III).

The above written description of the invention provides a manner and process of making and using it such that any person skilled in this art is enabled to make and use the same, this enablement being provided in particular for the subject matter of the appended claims, which make up a part of the original description including a composition comprising, in a physiologically acceptable medium, at least one C-glycoside compound and at least one of: ferulic acid, phloretin, caprylyl glycol, pomegranate extract, blueberry extract, sea fennel, retinol or one of its compounds, ginger extract, unsaturated omega-3 and/or omega-5 oil, spheres of collagen and of chondroitin sulphate, hyaluronic acid spheres, spring water from the Vichy basin, and fermented Alteromonas extract, and a composition comprising, in a physiologically acceptable medium, at least one C-glycoside compound and at least one foaming surfactant, said composition not comprising an oxyalkylenated compound.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising, in a physiologically acceptable medium, at least one C-glycoside compound and at least one selected from the group consisting of ferulic acid, phloretin, caprylyl glycol, pomegranate extract, blueberry extract, sea fennel, retinol or one of its compounds, ginger extract, unsaturated omega-3 and/or omega-5 oil, spheres of collagen and of chondroitin sulphate, hyaluronic acid spheres, spring water from the Vichy basin, and fermented Alteromonas extract.

2. The composition according to claim 1, comprising phloretin.

3. The composition according to claim 1, comprising caprylyl glycol.

4. The composition according to claim 1, comprising at least one pomegranate extract.

5. The composition according to claim 1, comprising at least one blueberry extract.

6. The composition according to claim 1, comprising sea fennel.

7. The composition according to claim 1, comprising retinol or one of its compounds.

8. The composition according to claim 1, comprising at least one ginger extract.

9. The composition according to claim 1, comprising unsaturated omega-3 and/or omega-5 oil.

10. The composition according to claim 1, comprising spheres of collagen and of chondroitin sulphate.

11. The composition according to claim 1, comprising hyaluronic acid spheres.

12. The composition according to claim 1, comprising spring water from the Vichy basin.

13. The composition according to claim 1, comprising at least one fermented Alteromonas extract.

14. The composition according to claim 1, comprising retinol palmitate.

15. The composition according to claim 1, comprising musk rose oil.

16. The composition according to claim 1, comprising ferulic acid.

17. The composition according to claim 1, comprising 0.0001% to 25% by weight relative to the total weight of the composition of at least one C-glycoside compound of formula (I): 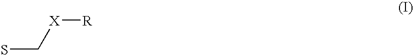
in which:
R represents:
a saturated C₁ to C₂₀ or unsaturated C₂ to C₂₀ linear alkyl radical, or a saturated or unsaturated C₃ to C₂₀ branched or cyclic alkyl radical; or
a saturated C₁ to C₂₀ or unsaturated C₂ to C₂₀ hydrofluoroalkyl or perfluoroalkyl radical, or a saturated or unsaturated C₃ to C₂₀ branched or cyclic hydrofluoroalkyl or perfluoroalkyl radical;
the hydrocarbon chain making up said radicals optionally being interrupted by 1, 2, 3 or more heteroatoms chosen from:
oxygen;
sulphur;
nitrogen; and
silicon,
and which may optionally be substituted by at least one radical chosen from:
—OR₄;
—SR₄;
—NR₄R₅;
—COOR₄;
—CONHR₄;
—CN;
a halogen atom;
a C₁ to C₆ hydrofluoroalkyl or perfluoroalkyl radical;
a C₃ to C₈ cycloalkyl radical;
with R₄ and R₅ representing, independently of one another, a hydrogen atom, or a saturated C₁ to C₃₀ or unsaturated C₂ to C₃₀ linear alkyl, perfluoroalkyl or hydrofluoroalkyl radical, or a saturated or unsaturated C₃ to C₃₀ branched or cyclic alkyl, perfluoroalkyl or hydrofluoroalkyl, or a C₆ to C₁₀ aryl radical,
X represents a radical chosen from: 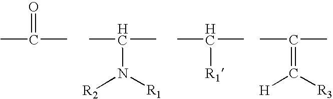
with R₁, R₂ and R₃ representing, independently of one another, a hydrogen atom or a radical R, with R as defined previously, R₁ also possibly representing a C₆ to C₁₀ aryl radical, and R₁′ represents a hydrogen atom, an —OH group or a radical R as defined previously;
S represents a monosaccharide or a polysaccharide composed of up to 20 sugar units in the form of pyranose and/or furanose and of L and/or D series, said monosaccharide or polysaccharide optionally being substituted by a compulsorily free hydroxyl group, and optionally one or more optionally protected amine functional groups; and
the S—CH₂—X linkage represents an anomeric C-type linkage, which may be α or β,
and cosmetically acceptable salts, solvates and isomers thereof.

18. The composition according to claim 1, comprising at least one C-glycoside compound selected from the group consisting of:
C-β-D-xylopyranoside-n-propan-2-one;
C-α-D-xylopyranoside-n-propan-2-one;
C-β-D-xylopyranoside-2-hydroxypropane;
C-α-D-xylopyranoside-2-hydroxypropane;
1-(C-β-D-fucopyranoside)propan-2-one;
1-(C-α-D-fucopyranoside)propan-2-one;
1-(C-β-L-fucopyranoside)propan-2-one;
1-(C-α-L-fucopyranoside)propan-2-one;
1-(C-β-D-fucopyranoside)-2-hydroxypropane;
1-(C-α-D-fucopyranoside)-2-hydroxypropane;
1-(C-β-L-fucopyranoside)-2-hydroxypropane;
1-(C-α-L-fucopyranoside)-2-hydroxypropane;
1-(C-β-D-glucopyranosyl)-2-hydroxypropane;
1-(C-α-D-glucopyranosyl)-2-hydroxypropane;
1-(C-(3-D-galactopyranosyl)-2-hydroxypropane;
1-(C-α-D-galactopyranosyl)-2-hydroxypropane;
1-(C-(3-D-fucofuranosyl)propan-2-one;
1-(C-α-D-fucofuranosyl)propan-2-one;
1-(C-β-L-fucofuranosyl)propan-2-one;
1-(C-α-L-fucofuranosyl)propan-2-one;
(C-β-D-maltopyranoside-n-propan-2-one;
(C-α-D-maltopyranoside-n-propan-2-one;
(C-β-D-maltopyranoside-2-hydroxypropane; and
(C-α-D-maltopyranoside-2-hydroxpropane.

19. A composition comprising, in a physiologically acceptable medium, at least one C-glycoside compound and at least one foaming surfactant, said composition not comprising an oxyalkylenated compound.

20. An assembly comprising:
a) a container delimiting at least one compartment, said container being sealed by a closure member; and
b) a composition placed inside said compartment, the composition being the composition according to claim 1.
